SEQUENCE LISTING (26 sequences)

SEQ ID NO: 1 - protein, 33 aa, Artificial
  GLP-2
HADGSFSDEMNTILDNLAARDFINWLIQTKITD

SEQ ID NO: 2 - protein, 30 aa, Artificial
  GLP-2
HAEGTFTSDVSSYLEGQAAKEFIAWLVKGR

SEQ ID NO: 3 - protein, 16 aa, Artificial
  Penetratin
RQIKIWFQNRRMKWKK

SEQ ID NO: 4 - protein, 10 aa, Artificial
  TAT
GRKKRRQRRR

SEQ ID NO: 5 - protein, 7 aa, Artificial
  Mini-Penetratin
RRMKWKK

SEQ ID NO: 6 - protein, 12 aa, Artificial
  R9FC
RRRRRRRRRFFC

SEQ ID NO: 7 - protein, 5 aa, Artificial
  AIP6
RLRWR

SEQ ID NO: 8 - protein, 16 aa, Artificial
  DPV3
RKKRRRESRKKRRRES

SEQ ID NO: 9 - protein, 17 aa, Artificial
  DPV6
GRPRESGKKRKRKRLKP

SEQ ID NO: 10 - protein, 21 aa, Artificial
  Pep-1
KETWWETWWTEWSQPKKKRKV

SEQ ID NO: 11 - protein, 27 aa, Artificial
  Transportan
GLAFLGFLGAAGSTMGAWSQPKKKRKV

SEQ ID NO: 12 - protein, 27 aa, Artificial
  MAP
GWTLNSAGYLLGKINLKALAALAKKIL

SEQ ID NO: 13 - protein, 17 aa, Artificial
  MAP
KLALKALKALKAALKLA

SEQ ID NO: 14 - protein, 9 aa, Artificial
  W/R
RRWWRRWRR

SEQ ID NO: 15 - protein, 20 aa, Artificial
  CADY
GLWRALWRLLRSLWRLLWRA

SEQ ID NO: 16 - protein, 22 aa, Artificial
  EB-1
LIRLWSHLIHIWFQNRRLKWKK

SEQ ID NO: 17 - protein, 10 aa, Artificial
  HRSV
RRIPNRRPRR

SEQ ID NO: 18 - protein, 13 aa, Artificial
  PTD-5
RRQRRRTSKLMKR

SEQ ID NO: 19 - protein, 11 aa, Artificial
  TAT47-57
YGRKKRRQRRR

SEQ ID NO: 20 - protein, 12 aa, Artificial
  TP2
PLIYLRLLRGQF

SEQ ID NO: 21 - protein, 21 aa, Artificial
  TP10
AGYLLGKINLHALAALAKKIL

SEQ ID NO: 22 - protein, 7 aa, Artificial
  PAS
FFLIPKG

SEQ ID NO: 23 - protein, 5 aa, Artificial
  PAS
LILIG

SEQ ID NO: 24 - skipped

SEQ ID NO: 25 - protein, 5 aa, Artificial
  PAS
FFFFG

SEQ ID NO: 26 - protein, 7 aa, Artificial
  PAS
FFFFFFG

(19) United States
(12) Patent Application Publication
(10) Pub. No.: US 2024/0270811 A1
(43) Pub. Date: Aug. 15, 2024

(54) Glycosylated Neuropeptide Derivative, Pharmaceutical Composition, Intranasal/Nasal Drop Formulation, and Use of Pharmaceutical Composition

(71) Applicant: Tokyo University of Science Foundation, Tokyo (JP)

(72) Inventor: Chikamasa Yamashita, Shinjuku-ku, Tokyo (JP)

(21) Appl. No.: 18/289,934

(22) PCT Filed: May 12, 2022
(86) PCT No.: PCT/JP2022/020095

§ 371 Date: Mar. 27, 2024

(30) Foreign Application Priority Data
May 13, 2021 (JP) 2021-081875

(51) Int. Cl. C07K 14/605; A61K 9/00; A61K 38/00; A61P 25/24; C07K 1/107

(52) U.S. Cl. CPC C07K 14/605; A61K 9/0043; A61P 25/24; C07K 1/1077; A61K 38/00

(57) ABSTRACT

A glycosylated neuropeptide derivative, having a neuropeptide sequence, a cell-penetration accelerating sequence, an endosomal-escape accelerating sequence, and a sugar chain.

### RELATED APPLICATIONS

This application is a National Stage Application under 35 U.S.C. 371 of co-pending PCT application PCT/JP2022/020095 designating the United States and filed May 12, 2022; which claims the benefit of JP application number 2021-081875 and filed May 13, 2021, each of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Apr. 26, 2018, is named “Replacement Sequence_007801.00040_ST25” and is 5.79 kb in size.

### TECHNICAL FIELD

The present invention relates to a glycosylated neuropeptide derivative, a pharmaceutical composition, an intranasal/nasal drop formulation, and a use of a pharmaceutical composition.

### BACKGROUND ART

Central nervous system diseases such as Alzheimer's disease, vascular dementia and amyotrophic lateral sclerosis are known to have a high degree of unmet medical needs, i.e., diseases with a low degree of medical satisfaction and an insufficient variety of effective drugs, and development of new drugs is required. On the other hand, a certain degree of therapeutic effects has been achieved by the use of low-molecular drugs for treating depression. However, since approximately 30-40% of patients exhibit treatment resistivity to existing anti-depressant drugs, development of drugs with a new medicinal mechanism is desired.

In view of the foregoing, neuropeptides, which express a different medical mechanism from that of low-molecular drugs, are attracting attention as possible alternative drugs. For example, it is known that glucagon-like peptide-1 (GLP-1), a peptide derived from a proglucagon and formed of 37 amino acid residues, and glucagon-like peptide-2 (GLP-2), a peptide derived from a proglucagon and formed of 33 amino acid residues, bind to a G-protein-coupled receptor (GPCR) and activate the signaling transduction.

Regarding the pharmacological activity of GLP-1 (activity types: 7-37 and 7-36 amide) in the brain, there have been reports of an effect of alleviating learning disorders (for example, Non-Patent Documents 1 and 2).

Regarding the pharmacological activity of GLP-2 in the brain, there have been reports of effects of anti-depressant activity even in treatment-resistant depression model animals, lowering blood pressure, and alleviating learning disorders (for example, Non-Patent Documents 3 to 9).

Further, it has been reported that neuromedin U (NmU), a peptide formed of 23 amino acid residues, binds to a GPCR in the brain and exerts an effect of alleviating learning disorders (for example, Non-Patent Document 10).

In addition, research and development have been conducted on centrally-acting peptides such as enkephalin (5 amino acid residues), pasircotide (5 amino acid residues), ocretide (7 amino acid residues), lanreotide (7 amino acid residues), oxytocin (9 amino acid residues), somatostatin-14 (14 amino acid residues), dynorphin (17 amino acid residues), somatostatin-28 (28 amino acid residues), ghrelin (28 amino acid residues), orexin B (28 amino acid residues), galanin (30 amino acid residues), β-endorphin (31 amino acid residues), orexin A (33 amino acid residues), neuropeptide Y (36 amino acid residues), insulin (51 amino acid residues), galanin-like peptide (60 amino acid residues), insulin-like growth factor-1 (70 amino acid residues), nerve growth factor (118 amino acid residues) and leptin (166 amino acid residues).

One significant factor behind a high degree of unmet medical needs in central nervous system diseases is difficulty in delivery of a drug to a target region due to tight cell-to-cell conjugation represented by blood-brain barrier (BBB), which makes a delivery route for a drug from blood to a brain tissue extremely restricted. For example, it is not possible for 100% of large molecules with a size of over 500 Da or not less than 98% of molecules smaller than the same to penetrate the BBB (Non-Patent Document 11). Therefore, a pharmaceutical test for a drug in central nervous system diseases is performed by way of intraventricular administration, i.e., injecting a drug directly into the brain. However, application of intraventricular injection to clinical practice is unrealistic due to its highly invasive nature. In view of the foregoing, intranasal administration has been attracting attention in which a drug is administered to the nasal cavity, which is anatomically located in the vicinity of the brain, as a non-invasive solution to the drug delivery system in consideration of clinical practice. In fact, there have been reports of animal experiments showing that many types of peptides are successfully delivered to the central nervous system via an olfactory bulb or a cerebral fluid (for example, Non-Patent Document 12). However, practical use of a peptide that exhibits a central action by way of intranasal administration has yet to be realized. One major reason for this is that the characteristics of the nasal mucosa have not been considered in the development of drug delivery systems (DDS).

The nasal mucosa is covered with an olfactory epithelium and a respiratory epithelium, and the human nasal mucosa consists of an olfactory epithelium approximately by 3% and a respiratory epithelium approximately by 97% (Non-Patent Document 13). Accordingly, delivering a peptide to the human central nervous system through the respiratory epithelium, rather than through the olfactory epithelium, would be an advantageous way of intranasal administration.

The following three routes are regarded as major delivery routes for an intranasally-administered drug to the central nervous system.
(1) A route in which a drug migrates to blood at nasal mucosa, and is delivered to the central nervous system by penetrating the BBB
(2) A route in which a drug migrates to an olfactory bulb at an olfactory epithelium, or disperses in a cerebral fluid through intracellular spaces at an olfactory epithelium, and is delivered to the central nervous system
(3) A route in which a drug is delivered to the central nervous system through a respiratory epithelium and trigeminal nerves

From the viewpoint of application of drugs to clinical practice, the route (3), taking advantage of human nasal mucosa characteristics as mentioned above, may be the most appropriate way to deliver a peptide to the central nervous system. However, it is reported that the lamina propria, a lower layer of the respiratory epithelium, has a high degree of vascular permeability due to an extremely large amount of blood capillaries existing therein, and a peptide passing through the intercellular spaces in the respiratory epithelium is absorbed in the whole body. For example, a nasal formulation of calcitonin is clinically used as a therapeutic drug for osteoporosis. The nasal formulation is designed such that calcitonin is absorbed in the whole body through the nasal mucosa by way of intranasal administration (Non-Patent Document 14).

Accordingly, in order to deliver a peptide to the central nervous system in an efficient manner, suppressing the penetration of a peptide through the intracellular spaces is an important issue. From this point of view, a nose-to-brain system that exhibits an action on the central nervous system has been proposed in which a neuropeptide derivative, obtained by adding a cell-penetration accelerating sequence and an endosomal-escape accelerating sequence to a neuropeptide, is delivered to an action site such as the hippocampus or the hypothalamus by way of intranasal administration (Patent Document 1).

### PRIOR ART DOCUMENTS

### Patent Document

Patent Document 1: International Publication No. 2016/035820

### Non-Patent Documents

Non-Patent Document 1: Neuroscience Research 64 (2009) 67-74
Non-Patent Document 2: Journal of Neuroscience Research 92 (2014) 446-454
Non-Patent Document 3: Behavioural Brain Research 204 (2009) 235-240
Non-Patent Document 4: Neuroscience 212 (2012) 140-148
Non-Patent Document 5: Life Sciences 93 (2013) 889-896
Non-Patent Document 6: Neuroscience Letters 550 (2013) 104-108
Non-Patent Document 7: Behavioural Brain Research 243 (2013) 153-157
Non-Patent Document 8: Neuropeptides 49 (2015) 7-14
Non-Patent Document 9: Neuroscience 294 (2015) 156-165
Non-Patent Document 10: Neuroscience Research 61 (2008) 113-119
Non-Patent Document 11: NeuroRx 2 (2005) 3-14
Non-Patent Document 12: European Journal of Pharmaceutical Sciences 40 (2010) 385-403
Non-Patent Document 13: Toxicologic Pathology 19 (1991) 321-336
Non-Patent Document 14: European Journal of Pharmaceutics and Biopharmaceutics 88 (2014) 8-27
Non-Patent Document 15: International Journal of Pharmaceutics 515 (2016) 37-45
Non-Patent Document 16: Nature 422 (2003) 37-44
Non-Patent Document 17: Neuroscientist 20 (2014) 71-81

### SUMMARY OF THE INVENTION

### Problem to be Solved by the Invention

The neuropeptide derivative obtained by adding a cell-penetration accelerating sequence and an endosomal-escape accelerating sequence to a neuropeptide described in Patent Document 1 is delivered to the central nervous system by intranasal administration and exhibits a central action. However, the neuropeptide derivative described in Patent Document 1 tends to be highly lipophilic and poorly soluble with respect to an aqueous medium. The reason for this may be that the neuropeptide derivative has a low degree of water solubility because a sequence containing highly hydrophobic amino acid residues, such as phenylalanine, is used for promoting the endosomal escape of the neuropeptide derivative. Therefore, the neuropeptide derivative is dissolved in 16% dimethylsulfoxide (DMSO) in the tests performed in the Examples described in Patent Document 1. However, since DMSO as an organic solvent is reported to have cellular cytotoxicity or stimulating properties for eyes or skins, the toxic nature of DMSO is an issue of concern in view of clinical application. Therefore, in order to productize the neuropeptide derivative as a clinical formulation, it is necessary to improve the solubility thereof with respect to an aqueous medium. As a means for improving the solubility of a drug that is poorly soluble with respect to an aqueous medium, additives such as a surfactant or an inclusion compound are generally used. However, there have been reports of cellular cytotoxicity caused by these additives (Patent Document 15).

Further, the neuropeptide derivative described in Patent Document 1 has room to improve in terms of drug retention in the central nervous system, continuity in drug efficacy, and enhancement in drug efficacy.

The present invention aims to provide a glycosylated neuropeptide derivative that exhibits excellent solubility with respect to an aqueous medium, drug retention in the central nervous system, continuity in drug efficacy, and enhancement in drug efficacy; and a pharmaceutical composition including the glycosylated neuropeptide derivative. Further, the present invention aims to provide an intranasal/nasal drop formulation including the glycosylated neuropeptide derivative, and a use of a pharmaceutical composition including the glycosylated neuropeptide derivative.

### Means for Solving the Problem

The means for solving the problem includes the following embodiments.
<1> A glycosylated neuropeptide derivative, having a neuropeptide sequence, a cell-penetration accelerating sequence, an endosomal-escape accelerating sequence, and a sugar chain.
<2> The glycosylated neuropeptide derivative according to <1>, wherein a number of monosaccharide residues per sugar chain is from 5 to 20.
<3> The glycosylated neuropeptide derivative according to <1> or <2>, wherein the sugar chain binds to the neuropeptide sequence.
<4> The glycosylated neuropeptide derivative according to any one of <1> to <3>, wherein a number of amino acid residues of the neuropeptide sequence is 200 or less.
<5> The glycosylated neuropeptide derivative according to any one of <1> to <4>, wherein the cell-penetration accelerating sequence is cationic.
<6> The glycosylated neuropeptide derivative according to any one of <1> to <5>, wherein half or more of a total number of amino acid residues of the cell-penetration accelerating sequence are basic amino acid residues.
<7> The glycosylated neuropeptide derivative according to any one of <1> to <6>, wherein the endosomal-escape accelerating sequence is an amino acid sequence selected from the group consisting of FFLIPKG, LILIG, FFG, FFFFG and FFFFFFG.
<8> The glycosylated neuropeptide derivative according to any one of <1> to <7>, wherein the glycosylated neuropeptide derivative reaches an action site via at least one of trigeminal nerve, trigeminal ganglion, principal sensory nucleus of trigeminal nerve or trigeminal lemniscus.
<9> The glycosylated neuropeptide derivative according to any one of <1> to <8>, having an ability for macropinocytosis.
<10> A pharmaceutical composition, comprising the glycosylated neuropeptide derivative according to any one of <1> to <9> as an active ingredient.
<11> The pharmaceutical composition according to <10>, used for therapy of a neuropsychiatric disorder or a neurodegenerative disorder.
<12> The pharmaceutical composition according to <10> or <11>, used for therapy of depression or dementia.
<13> An intranasal/nasal drop formulation, comprising the glycosylated neuropeptide derivative according to any one of <1> to <9> as an active ingredient.
<14> The intranasal/nasal drop formulation according to <13>, used for therapy of a neuropsychiatric disorder or a neurodegenerative disorder.
<15> The intranasal/nasal drop formulation according to <13> or <14>, used for therapy of depression or dementia.
<16> Use of a pharmaceutical composition for intranasal/nasal drop administration, the pharmaceutical composition comprising the glycosylated neuropeptide derivative according to any one of <1> to <9> as an active ingredient.

### Effect of the Invention

According to the present invention, it is possible to provide a glycosylated neuropeptide derivative that exhibits excellent solubility in an aqueous medium, drug retention in the central nervous system, continuity in drug efficacy, and enhancement in drug efficacy, and a pharmaceutical composition including the glycosylated neuropeptide derivative. Further, according to the present invention, it is possible to provide an intranasal/nasal drop formulation including the glycosylated neuropeptide derivative, and a use of a pharmaceutical composition including the glycosylated neuropeptide derivative.

### BRIEF EXPLANATION OF THE DRAWINGS

FIG. 1 is a graph showing the solubility of various GLP-2 derivatives in Example 1.

FIG. 2 is a graph showing the anti-depressant-like effects after intranasal administration of various GLP-2 derivatives in Example 2.

FIG. 3 is a graph showing the effect of PBS on anti-depressant-like effects of glycosylated GLP-2 derivatives (11-sugar) in Example 3.

FIG. 4 is an image obtained by optical imaging device showing the distribution of intranasally-administered PAS-CPP-GLP-2 derivative (no sugar) and PAS-CPP-GLP-2 derivative (11-sugar) in Example 4.

FIG. 5 is a graph showing the amount of PAS-CPP-GLP-2 derivative (no sugar) and PAS-CPP-GLP-2 derivative (11-sugar) delivered to the brain, quantified by ELISA in Example 5.

FIG. 6A is an image showing the distribution in the brain of PAS-CPP-GLP-2 derivative (no sugar) and PAS-CPP-GLP-2 derivative (11-sugar) obtained by immunostaining, 5 minutes after intranasal administration in Example 6.

FIG. 6B is an image showing the distribution in the brain of PAS-CPP-GLP-2 derivative (no sugar) and PAS-CPP-GLP-2 derivative (11-sugar) obtained by immunostaining, 20 minutes after intranasal administration in Example 6.

FIG. 7A is an image showing the distribution in the brain qualitatively and quantitatively, 5 minutes after intranasal administration in Example 7.

FIG. 7B is an image showing the distribution in the brain qualitatively and quantitatively, 20 minutes after intranasal administration in Example 7.

FIG. 7C is an image showing the distribution in the brain qualitatively and quantitatively, 60 minutes after intranasal administration in Example 7.

FIG. 8 is an image showing the localization in the trigeminal nerve of PAS-CPP-GLP-2 derivative (11-sugar), 5 minutes after intranasal administration in Example 8.

FIG. 9 is an image confirming the delivery to the trigeminal lemniscus of intranasally-administered glycosylated GLP-2 derivative in Example 9.

FIG. 10 is a graph showing the anti-depressant-like effects of PAS-CPP-GLP-2 derivative (11-sugar) and PAS-CPP-GLP-2 derivative (no sugar) after intranasal administration in Example 10.

FIG. 11 is a graph showing the anti-depressant-like effects of PAS-CPP-GLP-2 derivative and PAS-CPP-GLP-2 derivative (no sugar) after intranasal administration in Example 11.

FIG. 12 is a graph showing the engagement of micropinocytosis of PAS-CPP-GLP-2 derivative (11-sugar) in the cellular uptake mechanism into neurons cells Neuro A2 in Example 12.

FIG. 13A is a graph showing the effect of improvement in learning and memory functions of PAS-CPP-GLP-1 derivative (11-sugar) and PAS-CPP-GLP-1 derivative (no sugar) after intranasal administration in Example 13.

FIG. 13B is a graph showing the effect of improvement in learning and memory functions of PAS-CPP-GLP-1 derivative (no sugar) after intranasal administration or intraventricular administration.

FIG. 14 is a graph showing the usefulness of PAS-CPP in PAS-CPP-GLP-2 derivative.

### EMBODIMENTS FOR IMPLEMENTING THE INVENTION

In the following, embodiments of the invention are explained. The explanation and the embodiments described herein are intended to exemplify the invention, and do not limit the scope of the invention.

Numerical ranges indicated as “from A to B” described herein include A and B as a minimum value and a maximum value, respectively.

The left side of the amino acid sequence refers to the N-terminal, and the amino acid residue may be indicated by a one-letter abbreviation (for example, G for glycine residue) or a three-letter abbreviation (for example, Gly for glycine residue).

The term “treatment” as described herein refers not only to an activity or an effect that quenches or alleviates a symptom, but also to an activity or an effect that suppresses aggravation of the symptom.

The term “anti-depressant activity” or “anti-depressant effect” refers not only to an activity or an effect that quenches a symptom of depression, but also to an activity or an effect that suppresses aggravation of the symptom.

The term “learning disorder-alleviating activity” or “learning disorder-alleviating effect” refers not only to an activity or an effect that quenches a symptom of a learning disorder, but also to an activity or an effect that suppresses aggravation of the symptom.

### <Glycosylated Neuropeptide Derivative>

The glycosylated neuropeptide derivative of the present invention has a neuropeptide sequence, a cell-penetration accelerating sequence (hereinafter, also referred to as a cell penetrating peptide or CPP), an endosomal-escape accelerating sequence (hereinafter, also referred to as a penetration accelerating sequence or PAS), and a sugar chain.

As a solution for the problem of being poorly soluble with respect to an aqueous medium of neuropeptide derivatives, the inventor has produced a neuropeptide derivative to which a sugar chain is added (hereinafter, also referred to as a glycosylated neuropeptide derivative), without using an additive such as a surfactant or an inclusion compound that may cause damage to cells. Generally, neuropeptide derivatives have a problem of a trade-off between water solubility and membrane permeability. Therefore, there has been anxiety that while addition of a sugar chain to a neuropeptide derivative may improve the water solubility, it may increase the amount of the neuropeptide derivative required to exhibit pharmaceutical effects, or may cancel the pharmaceutical effects of the neuropeptide derivative.

However, the addition of a sugar chain to a neuropeptide derivative has resulted in surprising findings. Specifically, when a glycosylated neuropeptide derivative is administered in an intranasal manner, it is delivered to the central nervous system and remains in such an efficient manner that could not have been expected based on common knowledge, as compared with a neuropeptide derivative to which a sugar chain is not added. Further, the addition of a sugar chain to a neuropeptide derivative proves to be effective in increasing the continuity of pharmaceutical effects and enhancing the pharmaceutical effects.

A possible pathway for a glycosylated neuropeptide derivative to reach the central nervous system after being intranasally administered may be a pathway from the trigeminal nerve and trigeminal ganglion in the nasal cavity to the central nervous system such as the hippocampus or the hypothalamus, via the principal sensory nucleus of trigeminal nerve (Pr5) that exists at the pons in the brainstem.

A possible pathway from the principal sensory nucleus of trigeminal nerve to the central nervous system may be a trigeminal lemniscus. The trigeminal lemniscus may be referred to as the trigeminothalamic tract. In the present disclosure, the trigeminal lemniscus is a concept that includes the trigeminothalamic tract.

The glycosylated neuropeptide derivative of the present invention has a cell-penetration accelerating sequence and an endosomal-escape accelerating sequence. As shown in the Reference Example, while a derivative in which both of a cell-penetration accelerating sequence and an endosomal-escape accelerating sequence are added to a neuropeptide sequence exhibits an anti-depressant activity as a central activity, a derivative in which either one of a cell-penetration accelerating sequence or an endosomal-escape accelerating sequence is added to a neuropeptide sequence does not exhibit an anti-depressant activity. In other words, the achievement of excellent central activity by the glycosylated neuropeptide derivative of the present invention is attributable to the addition of both of a cell-penetration accelerating sequence and an endosomal-escape accelerating sequence.

The intended purpose of the glycosylated neuropeptide derivative is not particularly limited, as long as it makes use of a pharmacological activity expressed by the glycosylated neuropeptide derivative upon action on the central nervous system. Examples of the pharmacological activity include an anti-depressant activity, a learning disorder-alleviating activity, an anti-anxiety activity, a feeding suppression activity, a cognitive disorder-alleviating activity, a blood-pressure-lowering activity, an analgesic activity, a sleep-inducing activity, and an anti-epileptic activity. Therefore, the glycosylated neuropeptide derivative of the present invention is suitably used as a drug for the treatment of neuropsychiatric disorders or neurodegenerative disorders, such as an anti-depressant agent, a learning disorder-alleviating agent, an anti-anxiety agent, a feeding suppression agent, a cognitive disorder-alleviating agent, a blood-pressure-lowering agent, an analgesic agent, a sleep-inducing agent, and an anti-epileptic agent.

The number of amino acid residues included in the glycosylated neuropeptide derivative is not particularly limited. As shown in the Examples, it is confirmed that the glycosylated neuropeptide derivative is taken into nerve cells by way of macropinocytosis. The macropinocytosis is a system that causes cellular uptake by means of reconstruction of an actin skeleton and formation of a ruffling structure of fluid plasma membranes, and produces endosomal vesicles with a size of greater than 1 μm. Accordingly, it is expected that a glycosylated neuropeptide derivative with a large molecular size may be taken into a cell (Non-Patent Document 16).

For example, the total number of amino acid residues included in the glycosylated neuropeptide derivative may be determined depending on the total number of the neuropeptide sequence, cell-penetration accelerating sequence, endosomal-escape accelerating sequence and a spacer sequence. For example, the total number of amino acid residues included in the glycosylated neuropeptide derivative may be 250 or less, 200 or less, or 150 or less. The total number of amino acid residues included in the glycosylated neuropeptide derivative may be 10 or more, 20 or more, or 30 or more.

Each of the amino acid residues that constitutes the glycosylated neuropeptide derivative may be either L-form or D-form, as long as the effect of the invention is achieved. The method for producing the glycosylated neuropeptide derivative is not particularly restricted, and may be a method of extracting from a living body or a natural substance, or may be prepared by a genetic engineering process or an organic synthetic process.

### (Neuropeptide Sequence)

The neuropeptide sequence in the glycosylated neuropeptide sequence derivative is not particularly limited, as long as it is derived from a peptide that exhibits pharmaceutical effects by acting on the central nervous system. The method for adding a cell-penetration accelerating sequence, an endosomal-escape accelerating sequence and a sugar chain to a neuropeptide sequence is not particularly limited, and may be performed by a known method.

The number of amino acid residues included in the neuropeptide sequence in the glycosylated neuropeptide sequence derivative is not particularly limited, as long as the glycosylated neuropeptide derivative can be taken into cells by way of macropinocytosis, and may be determined in view of the nature of macropinocytosis. The total number of amino acid residues included in the neuropeptide sequence may be from 5 to 200, from 5 to 170, from 9 to 120, from 9 to 70, or from 9 to 60. The total number of amino acid residues included in the neuropeptide sequence may be from 5 or more, 10 or more, or 15 or more. The total number of amino acid residues included in the neuropeptide sequence may be 200 or less, 170 or less, 120 or less, 70 or less, 60 or less, or 51 or less.

In an embodiment, the neuropeptide sequence is an amino acid sequence derived from a neuropeptide having a centrally-acting activity.

Specific examples of the neuropeptide include GLP-1 (23 amino acid residues), GLP-2 (37 amino acid residues), enkephalin (5 amino acid residues), pasireotide (5 amino acid residues), oxytocin enkephalin (5 amino acid residues), ocretide (7 amino acid residues), lanreotide (7 amino acid residues), oxytocin (9 amino acid residues), somatostatin-14 (14 amino acid residues), dynorphin (17 amino acid residues), somatostatin-28 (28 amino acid residues), ghrelin (28 amino acid residues), orexin B (28 amino acid residues), galanin (30 amino acid residues), β-endorphin (31 amino acid residues), orexin A (33 amino acid residues), neuropeptide Y (36 amino acid residues), insulin (51 amino acid residues), galanin-like peptide (60 amino acid residues), insulin-like growth factor-1 (70 amino acid residues), nerve growth factor (118 amino acid residues), leptin (166 amino acid residues), dynorphin (17 amino acid residues), ghrelin (28 amino acid residues), orexin B (28 amino acid residues), galanin (30 amino acid residues), β-endorphin (31 amino acid residues), orexin A (33 amino acid residues), neuropeptide Y (36 amino acid residues), insulin (51 amino acid residues), galanin-like peptide (60 amino acid residues), insulin-like growth factor-1 (70 amino acid residues), nerve growth factor (118 amino acid residues) and leptin (166 amino acid residues).

In an embodiment of the neuropeptide derivative, the neuropeptide sequence is an amino acid sequence derived from a peptide that is any of the following (a1) to (a2) or (b).
(a1) a peptide formed of an amino acid sequence represented by

(GLP-2, SEQ ID NO: 1)
HADGSFSDEMNTILDNLAARDFINWLIQTKITD

(a2) a peptide formed of an amino acid sequence represented by

(GLP-1: active-type 7-36 amide, SEQ ID NO: 2)
HAEGTFTSDVSSYLEGQAAKEFIAWLVKGR-NH₂

(b) a peptide formed of an amino acid sequence represented by (a1) or (a2) in which one or several amino acid residues are deleted, replaced or added, the peptide having a centrally-acting activity.

Among these peptides, GLP-2 is known to exert an anti-depressant activity and a blood-pressure-lowering activity, and GLP-1 is known to exert a learning disorder-alleviating activity. Therefore, these peptides are useful as a neuropeptide sequence.

When a peptide is formed of an amino acid sequence of a specific peptide and an amino acid sequence of a different peptide, the term “amino acid sequence derived from a specific peptide” refers to a portion of the peptide corresponding to an amino acid sequence of the specific peptide.

When the neuropeptide sequence is derived from “(b) a peptide formed of an amino acid sequence represented by (a1) or (a2) in which one or several amino acid residues are deleted, replaced or added”, the number of the amino acid residues to be deleted, replaced or added is not particularly limited, as long as the neuropeptide sequence can exert the effect of the invention. For example, the number of the amino acid residues to be deleted, replaced or added may be from 1 to 10, preferably from 1 to 5, more preferably from 1 to 3.

### (Cell-Penetration Accelerating Sequence)

The cell-penetration accelerating sequence in the glycosylated neuropeptide derivative is an amino acid sequence derived from a cell-penetrating peptide, i.e., a peptide that exerts an activity to penetrate a cell membrane.

The configuration of the cell-penetrating peptide that constitutes the cell-penetration accelerating sequence is not particularly limited, as long as it has an activity to induce macropinocytosis.

Examples of the cell-penetrating peptide include oligoarginine (Rn, n is the number of arginine residues ranging from 6 to 12), oligolysine (Kn, n is the number of lysine residues ranging from 6 to 12), penetratin (RQIKIWFQNRRMKWKK, 16 amino acid residues, SEQ ID NO: 3), TAT (GRKKRRQRRR, 10 amino acid residues, SEQ ID NO: 4), mini penetratin (RRMKWKK, 7 amino acid residues, SEQ ID NO: 5), R9FC (RRRRRRRRRFFC, 12 amino acid residues, SEQ ID NO: 6), AIP6 (RLRWR, 5 amino acid residues, SEQ ID NO: 7), DPV3 (RKKRRRESRKKRRRES, 16 amino acid residues, SEQ ID NO: 8), DPV6 (GRPRESGKKRKRKRLKP, 17 amino acid residues, SEQ ID NO: 9), Pep-1 (KETWWETWWTEWSQPKKKRKV, 21 amino acid residues, SEQ ID NO: 10), MPG (GLAFLGFLGAAGSTMGAWSQPKKKRKV, 27 amino acid residues, sequence number 11), Transportan (GWTLNSAGYLLGKINLKALAALAKKIL, 27 amino acid residues, SEQ ID NO: 12), MAP (KLALKALKALKAALKLA, 17 amino acid residues, SEQ ID NO: 13), W/R (RRWWRRWRR, 9 amino acid residues, SEQ ID NO: 14), CADY (GLWRALWRLLRSLWRLLWRA, 20 amino acid residues, SEQ ID NO: 15), EB-1 (LIRLWSHLIHIWFQNRRLKWKK, 22 amino acid residues, SEQ ID NO: 16), HRSV (RRIPNRRPRR, 10 amino acid residues, SEQ ID NO: 17), PTD-5 (RRQRRRTSKLMKR, 13 amino acid residues, SEQ ID NO: 18), TAT47-57 (YGRKKRRQRRR, 11 amino acid residues, SEQ ID NO: 19), TP2 (PLIYLRLLRGQF, 12 amino acid residues, SEQ ID NO: 20), TP10 (AGYLLGKINLHALAALAKKIL, 21 amino acid residues, SEQ ID NO: 21), a cationic sequence that binds to heparan, a cationic sequence that binds to RNA, and a cationic sequence that binds to DNA.

While the cell-penetrating peptide that constitutes the cell-penetration accelerating sequence is not particularly limited as long as it has an activity to induce macropinocytosis, the cell-penetrating peptide is preferably a cell-penetrating peptide that is positively charged as a whole, or a cell-penetrating peptide that is cationic as a whole. For example, a cationic cell-penetrating peptide formed of an amino acid sequence that is rich in basic amino acid residues such as arginine, lysine, histidine or tryptophan (for example, an amino acid sequence in which half or more of the total amino acid residues are basic amino acid residues) is preferred. Examples of such cell-penetrating peptides include oligoarginine (Rn, n is the number of arginine residues ranging from 6 to 12), TAT derived from a Tat protein of human immunodeficiency virus 1 (HIV-1), penetratin, Pep-1, MPG, MAP, CADY, EB-1 and Transportan.

It is thought that a cell-penetrating peptide formed of an amino acid sequence that is rich in basic amino acid residues induces macropinocytosis as a form of endocytosis, which is a process of a cell to take in an extracellular substance. As such, it is thought that the glycosylated neuropeptide derivative is introduced into a cell in a more efficient manner.

While the cell-penetration accelerating sequence is not particularly limited as long as it has an activity to induce macropinocytosis, the number of amino acid residues thereof is preferably from 5 to 27. Further, the cell-penetration accelerating sequence is preferably a peptide in which half or more of the total amino acid residues are basic amino acid residues; more preferably a peptide that includes arginine residues as the basic amino acid residues; further preferably an oligoarginine formed of 6 to 12 arginine residues; yet further preferably an oligoarginine formed of 7 to 9 arginine residues; yet further preferably an oligoarginine formed of 8 arginine residues.

### (Endosomal-Escape Accelerating Sequence)

It is thought that the endosomal-escape accelerating sequence shortens the time for the glycosylated neuropeptide derivative, which has been introduced into a cell, to remain in an endosome, and allows the glycosylated neuropeptide derivative to escape from an endosome in a shorter period. As a result, it is thought that the delivery of the glycosylated neuropeptide derivative to the central nervous system and the distribution of the glycosylated neuropeptide derivative in the central nervous system can be achieved in a shortened period.

The structure of the endosomal-escape accelerating sequence is not particularly limited, and examples thereof include amino acid sequences having an activity to promote endosomal escape such as FFLIPKG (SEQ ID NO: 22), LILIG (SEQ ID NO: 23), FFG (SEQ ID NO: 24), FFFFG (SEQ ID NO: 25) and FFFFFFG (SEQ ID NO: 26).

The position of the cell-penetration accelerating sequence and the endosomal-escape accelerating sequence in the glycosylated neuropeptide derivative is not particularly limited. For example, cither one of the cell-penetration accelerating sequence or the endosomal-escape accelerating sequence may be disposed at a position closer to the neuropeptide sequence.

From the viewpoint of achieving the effect of the invention in a more efficient manner, it is preferred that the cell-penetration accelerating sequence is disposed at a position closer to the neuropeptide sequence; and it is more preferred that the cell-penetration accelerating sequence is disposed at a position closer to the neuropeptide sequence and the endosomal-escape accelerating sequence is disposed at a N-terminal side or a C-terminal side of the cell-penetration accelerating sequence.

### (Sugar Chain)

The type of the sugar chain in the glycosylated neuropeptide derivative is not particularly limited. Specific examples of the sugar chain include N-linked sugar chains of high-mannose type, composite type or hybrid type (combination of high-mannose type and composite type), O-linked sugar chains, and proteoglycans such as mucin-type proteoglycan, heparan sulfate, chondroitin sulfate, keratan sulfate, hyaluronic acid and dermatan sulfate. Among these sugar chains, N-linked sugar chains are preferred and composite-type N-linked sugar chains are more preferred.

The configuration of the sugar chain is not particularly limited, and may be a biantennary structure or other configurations (such as a branched configuration). Examples of the monosaccharide that constitutes a sugar chain include glucose, mannose, galactose, fructose, N-acetylglucosamine, N-acetylgalactosamine, N-acetylmannosamine, fucose, sialic acid, N-acetylneuramic acid, N-glycolylneuramic acid, deaminoncuramic acid, glucuronic acid, iduronic acid, galacturonic acid, xylose, ribose and deoxyribose. The monosaccharide that constitutes a sugar chain may be either D-form or L-form. The monosaccharide that constitutes a sugar chain may be either an α-anomer or a β-anomer.

The number of the sugar chain in the glycosylated neuropeptide derivative may be one or more than one.

In the present disclosure, the number of the sugar chain is counted based on a basal portion of the sugar chain. Specifically, a group of monosaccharides stemming from a single basal portion is regarded as a single sugar chain.

The sugar chain may bind to an amino acid residue that constitutes the glycosylated neuropeptide derivative in a direct manner or an indirect manner.

In the present disclosure, each of a state in which a sugar chain directly binds to an amino acid residue that constitutes the glycosylated neuropeptide derivative, or a state in which a sugar chain indirectly binds to an amino acid residue that constitutes the glycosylated neuropeptide derivative, is regarded as a state in which a sugar chain binds to an amino acid residue that constitutes the glycosylated neuropeptide.

Examples of a state in which a sugar chain indirectly binds to an amino acid residue that constitutes the glycosylated neuropeptide derivative include a state in which a sugar chain binds to an amino acid residue that constitutes the glycosylated neuropeptide derivative via an amino acid residue such as a cysteine residue or an asparagine residue.

It is preferred that the sugar chain binds to a portion such that the functions of the cell-penetration accelerating sequence or the endosomal-escape accelerating sequence are favorably maintained, and it is more preferred that the sugar chain binds to the neuropeptide sequence.

When the sugar chain binds to the neuropeptide sequence, the binding site for the sugar chain is not particularly limited, as long as the stability or activity of the neuropeptide is not lowered. Specifically, the binding site for the sugar chain may be any of C-terminal side, N-terminal side, or other portions of the neuropeptide.

It is possible to substitute a portion of the neuropeptide sequence with an amino acid residue to which a sugar chain easily binds, such as a cysteine residue or an asparagine residue, as long as the bioactivity of the neuropeptide is not affected.

The binding site for the sugar chain to the neuropeptide sequence is preferably apart from the cell-penetration accelerating sequence and the endosomal-escape accelerating sequence, such that the functions thereof are not affected. The binding site for the sugar chain to the neuropeptide sequence is not particularly limited, as long as the functions of the cell-penetration accelerating sequence and the endosomal-escape accelerating sequence are not affected and the bioactivity of the neuropeptide is not affected. For example, when the cell-penetration accelerating sequence and the endosomal-escape accelerating sequence are added to an N-terminal side of the neuropeptide sequence, the sugar chain preferably binds to a C-terminal side of the neuropeptide sequence. When the cell-penetration accelerating sequence and the endosomal-escape accelerating sequence are added to a C-terminal side of the neuropeptide sequence, the sugar chain preferably binds to an N-terminal side of the neuropeptide sequence.

The number of monosaccharide residues per sugar chain is not particularly limited, and may be from 5 to 20 or from 5 to 15, for example.

The research conducted by the inventor has proved that the addition of a sugar chain consisting of a certain number of monosaccharide residues improves the solubility of the neuropeptide derivative with respect to an aqueous medium. Specifically, the number of monosaccharide residues per sugar chain is preferably 5 or more, more preferably 10 or more.

### (Spacer Sequence)

In the glycosylated neuropeptide derivative, the neuropeptide sequence and the cell-penetration accelerating sequence or the endosomal-escape accelerating sequence may be directly linked to each other, or a spacer sequence may be disposed therebetween. When a spacer sequence is disposed between the neuropeptide sequence and the cell-penetration accelerating sequence or the endosomal-escape accelerating sequence, the spacer sequence may exert an effect of preventing the activity of the neuropeptide sequence from lowering or deteriorating.

Generally, the cell-penetration accelerating sequence is formed of basic amino acid residues. Therefore, when the neuropeptide sequence includes acidic amino acid residues, it may be possible to prevent the cell-penetration accelerating sequence and the neuropeptide sequence from interacting with each other, and avoid the lowering or degradation of the activity of the neuropeptide sequence, by disposing a spacer sequence including neutral amino acid residues such as glycine by the number of from 1 to 10, preferably from 2 to 6.

In the glycosylated neuropeptide derivative, the neuropeptide sequence and the sugar chain may be directly linked to each other or a spacer sequence may be disposed therebetween. The type of the amino acid residue for the spacer sequence is not particularly limited, as long as an amino acid residue to which a sugar chain easily binds is introduced therein.

The glycosylated neuropeptide derivative may be subjected to various kinds of modification according to the intended purposes, in addition to the addition of a sugar chain. Examples of the modification include amino group modification (such as biotinylation, myristoylation, palmitoylation, acetylation and malcimidation), carboxy group modification (such as amidation and esterification), thiol group modification (such as farnesylation, geranylation, methylation and palmitoylation), hydroxy group modification (such as phosphorylation, sulfation, octanoylation, palmitoylation and palmitoleoylation, fluorescence labelling (such as FITC, FAM, rhodamine, BODIPY, NBD and MCA), pegylation, and introduction of amino acids such as an unnatural amino acid or a D-amino acid. The modification may be performed on any of the neuropeptide sequence, cell-penetration accelerating sequence, endosomal-escape accelerating sequence or spacer sequence.

The combination of the neuropeptide sequence, cell-penetration accelerating sequence and endosomal-escape accelerating sequence as the constituents of the glycosylated peptide derivative is not particularly limited, and may be selected depending on the intended purposes.

In an embodiment, the glycosylated peptide derivative may have an endosomal-escape accelerating sequence, a cell-penetration accelerating sequence, a spacer sequence as necessary, and a neuropeptide sequence, in this order from the N-terminal side.

In an embodiment, the glycosylated peptide derivative may have an endosomal-escape accelerating sequence, a cell-penetration accelerating sequence, a spacer sequence as necessary, and a neuropeptide sequence, in this order from the C-terminal side.

In the aforementioned configurations, the endosomal-escape accelerating sequence may be selected from FFLIPKG, LILIG, FFG, FFFFG or FFFFFFG.

In the aforementioned configurations, the cell-penetration accelerating sequence may be selected from oligoarginines (Rn, n=6-12).

In the aforementioned configurations, the spacer sequence may be selected from glycine residues (Gn, n=2-6).

### <Pharmaceutical Composition>

The pharmaceutical composition of the present invention includes a glycosylated neuropeptide derivative as an active ingredient. By including a glycosylated neuropeptide derivative as an active ingredient, the pharmaceutical composition can be rapidly delivered to the central nervous system and express a pharmacological effect efficiently. Therefore, the pharmaceutical composition is suitable for the therapy of diseases in which daily medication at home is required. Accordingly, preferred examples of formulation of the pharmaceutical composition include intranasal/nasal formulations.

The neuropsychiatric disorder or neurodegenerative disorder as a therapeutic objective for the pharmaceutical composition is not particularly limited, as long as the neuropeptide sequence in the glycosylated neuropeptide derivative acts on the central nervous system and exerts therapeutic effects on the therapeutic objective.

Examples of the neuropsychiatric disorders or neurodegenerative disorders as a therapeutic objective include depression, learning disorder, anxiety, eating disorder, cognition disorder, high blood pressure, sleeping disorder, epilepsia, Alzheimer's disease, vascular dementia, and amyotrophic lateral sclerosis.

Specific examples of the pharmaceutical composition include an anti-depressant agent, a learning disorder-alleviating agent, an anti-anxiety agent, a feeding suppression agent, a cognitive disorder-alleviating agent, a blood-pressure-lowering agent, an analgesic agent, a sleep-inducing agent, and an anti-epileptic agent. The type of the neuropeptide sequence in the glycosylated neuropeptide derivative may be selected according to the therapeutic objective.

For example, a pharmaceutical composition including a glycosylated neuropeptide derivative having a neuropeptide sequence derived from GLP-2 as an active ingredient is effective as an anti-depressant agent. Further, since GLP-2 exerts a blood pressure-lowering activity, a pharmaceutical composition including a glycosylate neuropeptide having a neuropeptide sequence derived from GLP-2 is thought to be particularly effective for cases expressing symptoms of depression due to a strong pressure and high blood pressure in combination. A pharmaceutical composition including a glycosylated neuropeptide derivative having a neuropeptide sequence derived from GLP-1 as an active ingredient thought to be effective as a learning disorder-alleviating agent, and is expected as a therapeutic agent for dementia.

The details and the preferred embodiments of the glycosylated neuropeptide derivative included in the pharmaceutical composition are as described above. From the viewpoint of the ability to be delivered to the central nervous system, the method for using the pharmaceutical composition is preferably intranasal/nasal administration.

The pharmaceutical composition may include a component other than the glycosylated neuropeptide derivative. Examples of the component other than the glycosylated neuropeptide derivative include a medium and a formulation additive that are used for the preparation of a pharmaceutical composition. Specific examples of the formulation additive include a diluent, a disintegrant, a binder, a lubricant, a surfactant, a buffer, a solubilizing agent, a stabilizer, a tonicity agent, a suspending agent, an emulcifier, a solvent, a thickner, a mucolytic agent, a humectant and a preservative. The dosage amount of the pharmaceutical composition may be selected according to the type of disease, the symptomatic state, weight or age of the patient, the administration form and the like.

The pharmaceutical composition of the present invention is particularly suitable as an intranasal/nasal formulation. Therefore, an embodiment of the present invention is a use of the present invention for intranasal/nasal administration.

### <Intranasal/Nasal Formulation>

The intranasal/nasal formulation of the present invention includes a glycosylated neuropeptide derivative as described above as an active ingredient. By including a glycosylated neuropeptide derivative as an active ingredient, the intranasal/nasal formulation can be rapidly delivered to the central nervous system and efficiently exerts a pharmacological effect. Further, since the intranasal/nasal formulation can be administered is a less invasive manner, it is useful for the therapy of a disease that requires daily medication at home.

The intranasal/nasal formulation may include a component other than the glycosylated neuropeptide derivative. Examples of the component other than the glycosylated neuropeptide derivative include a medium and a formulation additive that may be used for the preparation of the pharmaceutical composition, such as those as descried above.

The embodiments of the present invention include a use of the pharmaceutical composition including a glycosylated neuropeptide derivative as an active ingredient, as described above, for intranasal administration. The details and the preferred embodiments of the glycosylated neuropeptide derivative for the use are as described above.

### <Therapeutic Method for Neuropsychiatric Disorders or a Neurodegenerative Disorders>

The embodiments of the present invention include a therapeutic method for neuropsychiatric disorders or a neurodegenerative disorders, the method including administrating, to a patient, the glycosylated neuropeptide derivative or the pharmaceutical composition as mentioned above. The details and preferred embodiments of the glycosylated neuropeptide derivative or the pharmaceutical composition in the therapeutic method are as described above.

Specific examples of the neuropsychiatric disorders or a neurodegenerative disorders to be treated by the therapeutic method include depression, learning disorder, anxiety, eating disorder, cognition disorder, high blood pressure, sleeping disorder, epilepsia, Alzheimer's disease, vascular dementia, and amyotrophic lateral sclerosis.

While the method for administrating the glycosylated neuropeptide derivative or the pharmaceutical composition to a patient is not particularly limited, it is preferably intranasal administration.

### EXAMPLES

The present invention are explained in further detail by referring to the following examples. The materials, amounts, rates, procedures and the like may be modified without departing from the scope of the invention. Therefore, the scope of the present invention should not be construed in a restricted manner by the examples as described below.

### <Preparation of Glycosylated GLP-2 Derivative>

A glycosylated GLP-2 derivative referred to as PAS-CPP-GLP-2 (11-sugar), in which an endosomal-escape accelerating sequence (PAS: FFLIPKG), a cell-penetration accelerating sequence (CPP: RRRRRRRR), a spacer sequence (GG) and an amino acid sequence derived from GLP-2 as a neuropeptide sequence are disposed in this order from the N-terminal side, was prepared by an ordinary process. The neuropeptide sequence used for the preparation was added with a molecule including a sugar chain consisting of 11 monosaccharide residues, at the C-terminal of GLP-2 via a cysteine residue. The configuration of the glycosylated GLP-2 derivative is described below.

A fluorescent-labeled PAS-CPP-GLP-2 (11-sugar) used in some experiments was prepared by adding a fluorescent label (FITC or ICG) to the endosomal-escape accelerating sequence in PAS-CPP-GLP-2 (11-sugar).

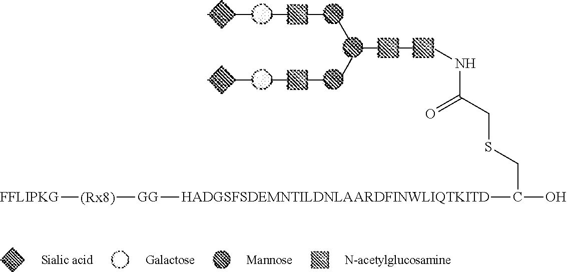

A glycosylated GLP-2 derivative referred to as PAS-CPP-GLP-2 (5-sugar) was prepared in the same manner as PAS-CPP-GLP-2 (11-sugar), except that a neuropeptide sequence added with a molecule including a sugar chain consisting of 5 monosaccharide residues was used. The configuration of the glycosylated GLP-2 derivative is described below.

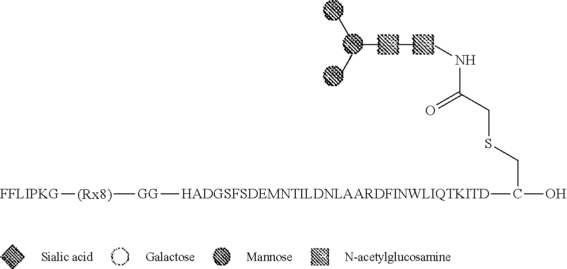

A GLP-2 derivative being not glycosylated referred to as PAS-CPP-GLP-2 (no sugar) was prepared in the same manner as PAS-CPP-GLP-2 (11-sugar), except that a neuropeptide sequence added with a molecule not including a sugar chain at the C-terminal of GLP-2 via a cysteine residue was used. The configuration of the GLP-2 derivative is described below.

A fluorescent-labeled PAS-CPP-GLP-2 (no sugar) used in some experiments was prepared by adding a fluorescent label (FITC or ICG) to the endosomal-escape accelerating sequence in PAS-CPP-GLP-2 (no-sugar).

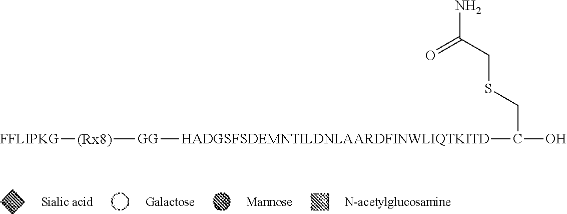

### Example 1: Evaluation on Solubility of GLP-2 Derivative with Respect to Aqueous Medium

Samples were prepared by dispensing a Milli-Q aqueous solution of PAS-CPP-GLP-2 (no sugar), PAS-CPP-GLP-2 (5-sugar) or PAS-CPP-GLP-2 (11-sugar) to a microtube by an amount of 5 nmol/tube, 30 nmol/tube, 60 nmol/tube, 120 nmol/tube or 200 nmol/tube, and freeze-drying the same. The freeze-dried samples were added with 200 μm of PBS (Dulbecco's Phosphate Buffered Saline; Sigma-Aldrich, herein after the same), subjected to ultrasonic treatment, and allowed to stand overnight. Thereafter, the concentration of the peptide was measured. Specifically, a supernatant obtained by centrifuging the sample suspension was subjected to the measurement of OD (280 nm) with a spectrometer (NanoDrop™ 2000c spectrometer; Thermo Fisher Scientific K.K.)

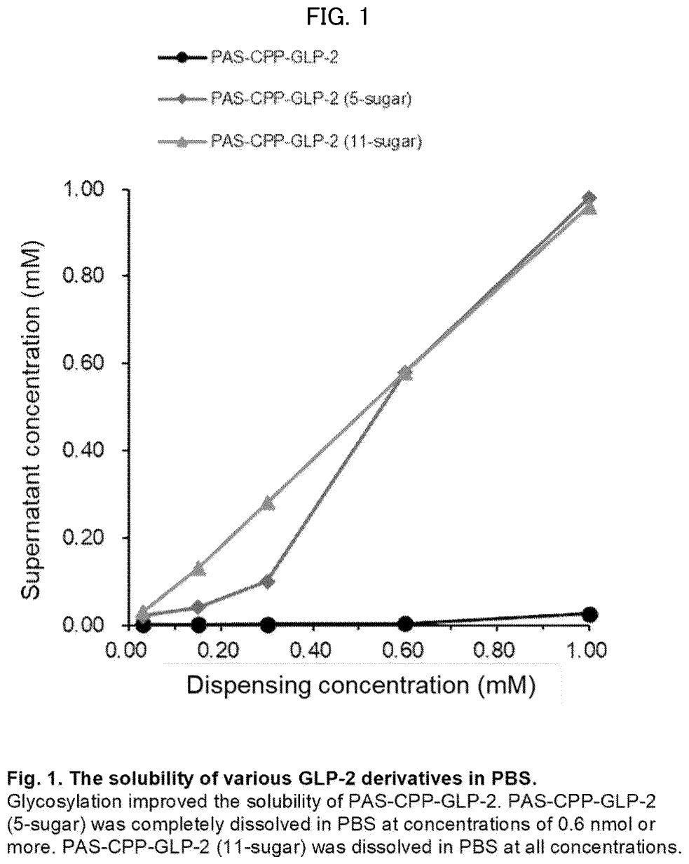

As shown in FIG. 1, PAS-CPP-GLP-2 derivative (no sugar) was not dissolved in PBS at all. Each of the glycosylated GLP-2 derivatives (5-sugar or 11-sugar) exhibited an improved solubility with respect to PBS. In particular, the solubility of GLP-2 derivatives (11-sugar) was linearly improved.

### Example 2: Evaluation on Influence of Glycosylation on Pharmaceutical Effects of GLP-2 Derivative

A forced swim test (FST) was performed on mice in order to evaluate the influence of glycosylation on anti-depressant-like effects of GLP-2 derivatives (11-sugar, 5-sugar).

### (Preparation and Administration of Administration Solution)

The following test was performed to evaluate whether or not the pharmaceutical effects are maintained by glycosylation.

Administration solutions were prepared by completely dissolving PAS-CPP-GLP-2 derivative (no sugar) or PAS-CPP-GLP-2 derivatives (11-sugar, 5-sugar) in DMSO and adding PBS such that the final concentration of DMSO was 16% by mass. The concentration of each GLP-2 derivative was adjusted to 0.6 nmol/4 μL.

After anesthetizing mice with isoflurane using an all-in-one small animal anesthetizer (MK-AT210D, Muromachi Kikai Co., Ltd., hereinafter the same), the solution was intranasally administered to the mice. Specifically, the intranasal administration was performed by contacting a tip of the anesthetizer to a nasal cavity in a horizontal manner such that the solution was inhaled by spontaneous respiration, by the amount of 2 μL per nose cavity, 4 μL in total (0.6 nmol/mouse). To the control group (Vehicle), a PBS solution containing DMSO by 16% by mass was intranasally administered by the amount of 4 μL.

The intranasal administration was performed 20 minutes before a test session of a forced swim test (FST) as set forth below.

### (Forced Swim Test)

A 7-week-old male ddY mouse is placed in a transparent plastic cylinder with a diameter of 18 cm and a height of 50 cm containing water at a temperature of 25° C.±1° C. to a height of 7 cm, and is allowed to swim for 15 minutes. Thereafter, the mouse is taken out from the cylinder and dried off with a towel, and returned to a cage. The procedure as mentioned above is referred to as a training session. After 24 hours of the completion of the training session, a 15-minute test session is performed in the same manner to the training session. The behavior of the mouse is recorded over the test session with a video camera. When the mouse is placed in the cylinder, it struggles to try to escape. When the mouse realizes that it cannot escape, the mouse gradually ceases to move giving up the escape. This situation is referred to as a state of immobility in which the mouse is in a depression-like state. The time during which the mouse is in a state of immobility (immobility time) is measured in the first 6 minutes from the start of the test session. The existence or non-existence of anti-depressant-like effects is determined by the length of the immobility time.

In the Examples described in the present specification, each of the forced swim is performed by the aforementioned method.

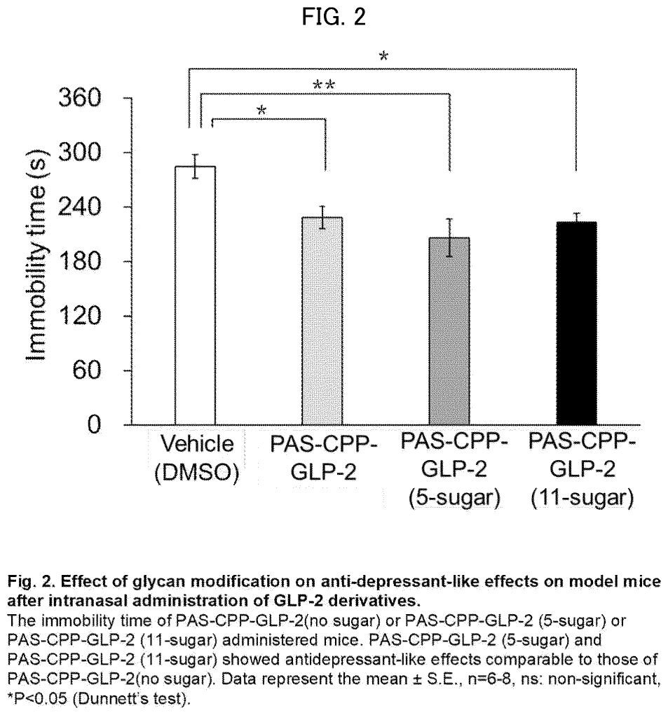

As shown in FIG. 2, the group administered with GLP-2 derivatives exhibited a significantly shortened immobility time as compared with the control group, indicating the expression of anti-depressant effects, irrespective of the existence or non-existence of glycosylation.

The results suggest that a sugar chain (11-sugar) binding at the C-terminal of GLP-2 does not affect the pharmaceutical effects of PAS-CPP-GLP-2 derivative.

### Example 3: Evaluation on Influence of PBS on Pharmaceutical Effects of Glycosylated GLP-2 Derivative

A forced swim test (FST) was performed on mice in order to evaluate the influence of PBS on anti-depressant-like effects of a glycosylated GLP-2 derivative (11-sugar).

### (Preparation and Administration of Administration Solution)

Administration solutions were prepared by mixing a PAS-CPP-GLP-2 derivative (no sugar) or a PAS-CPP-GLP-2 derivative (11-sugar) with PBS. In the solutions, the derivative with no sugar was suspended and the derivative with 11-sugar was completely dissolved. The concentration of each GLP-2 derivative was adjusted to 0.6 nmol/4 μL.

After anesthetizing mice with isoflurane using an all-in-one small animal anesthetizer (MK-AT210D, Muromachi Kikai Co., Ltd., hereinafter the same), the solution was intranasally administered to the mice by the amount of 2 μL per nose cavity, 4 μL in total (0.6 nmol/mouse). To the control group (Vehicle), an equivalent amount of DMSO by 16% by mass was intranasally administered.

The intranasal administration was performed 20 minutes before a test session of a forced swim test (FST) as set forth below.

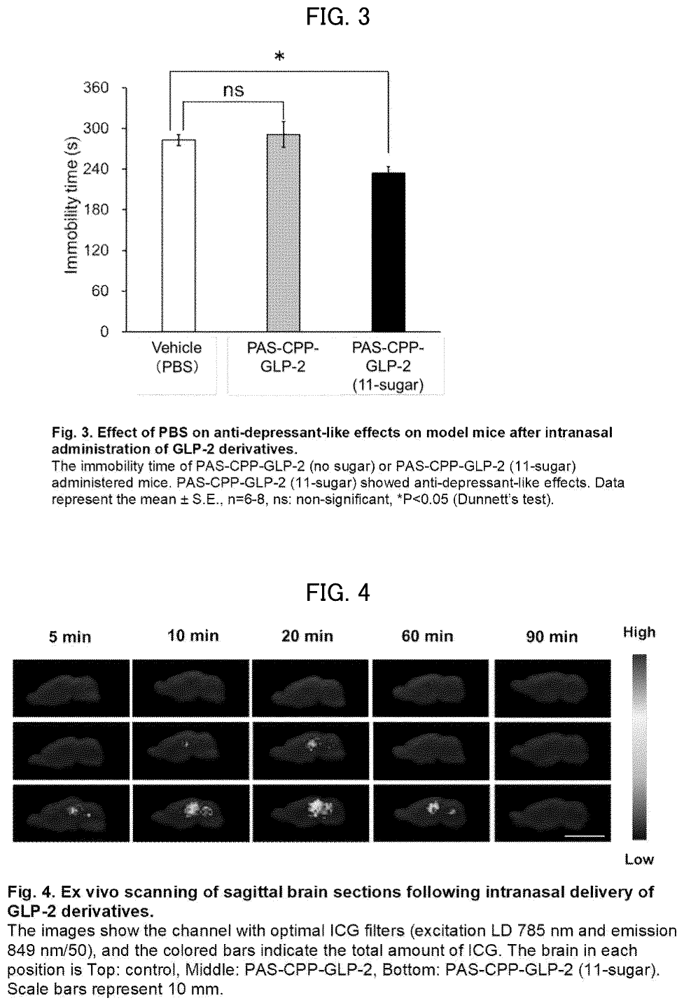

As shown in FIG. 3, the group administered with a PAS-CPP-GLP-2 derivative (no sugar) did not exhibit a significant anti-depressant-like effect as compared with the control group. The cause for the result is thought to be that the PAS-CPP-GLP-2 derivative did not dissolve in PBS. The group administered with PAS-CPP-GLP-2 derivative (11-sugar) exhibited a significant anti-depressant-like effect as compared with the control group. The cause for the result is thought to be that the PAS-CPP-GLP-2 derivative dissolved in PBS by mean of glycosylation.

The results suggest that the glycosylated PAS-CPP-GLP-2 derivative has an ability to express a centrally-acting activity even with an aqueous medium such as PBS. Accordingly, it is proved that there is no problem of initial concern, i.e., pharmaceutical effects may deteriorate due to an insufficient cell permeability of a derivative, due to an increase in water solubility caused by glycosylation.

### Example 4: Evaluation on Influence of Glycosylation on Migration of GLP-2 Derivative to Central Nervous System

The state of migration of GLP-2 derivatives to the central nervous system was analyzed with an optical imaging apparatus in order to evaluate the influence of glycosylation on an ability of the GLP-2 derivative to migrate to the central nervous system.

### (Preparation of Administration Solution)

Administration solutions were prepared by completely dissolving a PAS-CPP-GLP-2 derivative (no sugar) labelled with ICG or a PAS-CPP-GLP-2 derivative (11-sugar) labelled with ICG in DMSO, and adjusting the final concentration of DMSO to 16% by adding PBS.

While a PAS-CPP-GLP-2 derivative (11-sugar) was completely soluble in PBS, DMSO was used as a medium in order to conform the conditions to that of a PAS-CPP-GLP-2 derivative (no sugar). The concentration of a PAS-CPP-GLP-2 derivative (no sugar) or a PAS-CPP-GLP-2 derivative (11-sugar) was adjusted to 3.0 nmol/4 μL.

(Evaluation on Pathway to Central Nervous System with Optical Imaging Apparatus)

After anesthetizing mice with isoflurane using an all-in-one small animal anesthetizer, the administration solutions or 16% DMSO as a control was intranasally administered to the mice by the amount of 2 μL per nose cavity, 4 μL in total.

In order to observe the migration to the brain over time, the brain was isolated 5 minutes, 10 minutes, 20 minutes, 60 minutes and 90 minutes after the intranasal administration, respectively, and subjected to infiltration fixation with 4% paraformaldehyde (4% PFA) solution overnight. Subsequently, a sagittal section with a thickness of 2 mm at the left and the right from the center of the brain was prepared using a brain matrix (RBM-2000S, ASI). The section was placed in a dish and analyzed with an optical imaging apparatus (Clairvivo OPT plus, Shimadzu Corporation) at an excitation wavelength of 785 nm, fluorescence wavelength of 849 nm, and an exposure time: 6 seconds.

The results are shown in FIG. 4. Surprisingly, fluorescence was observed only in the case administered with a PAS-CPP-GLP-2 derivative (11-sugar) (Bottom) at a region around a posterior portion of the brain and the hippocampus/hypothalamic area after 5 minutes of the intranasal administration.

After 10 minutes from the intranasal administration, fluorescence was observed in each of the case administered with a PAS-CPP-GLP-2 derivative (no sugar) (Middle) and the case administered with a PAS-CPP-GLP-2 derivative (11-sugar). In the case administered with a PAS-CPP-GLP-2 derivative (no sugar), fluorescence was observed at a region around the hippocampus. In the case administered with a PAS-CPP-GLP-2 derivative (11-sugar), fluorescence was observed intensively at a region at which fluorescence was observed 5 minutes after the intranasal administration, with an increased intensity.

After 20 minutes from the intranasal administration, fluorescence was observed in each of the case administered with a PAS-CPP-GLP-2 derivative (no sugar) and the case administered with a PAS-CPP-GLP-2 derivative (11-sugar). Fluorescence was observed in each case at a region around the hippocampus with an increased intensity as compared with 10 minutes after the administration, especially in the case of PAS-CPP-GLP-2 derivative (11-sugar).

After 60 minutes from the intranasal administration, while fluorescence was not observed in the case administered with a PAS-CPP-GLP-2 derivative (no sugar), a relatively strong degree of fluorescence was observed in the case administered with a PAS-CPP-GLP-2 derivative (11-sugar).

After 90 minutes of the intranasal administration, fluorescence was not observed even in the case administered with a PAS-CPP-GLP-2 derivative (11-sugar), suggesting the extinction thereof in the brain.

The results prove that a PAS-CPP-GLP-2 derivative (11-sugar) remains in the brain for a longer time period than a PAS-CPP-GLP-2 derivative (no sugar).

### Example 5: Effect of Glycosylation on Amount of Migration of GLP-2 Derivative to Brain

The results of Example 4 prove that a greater amount of a PAS-CPP-GLP-2 derivative (11-sugar) migrates to the brain as compared with a PAS-CPP-GLP-2 (no sugar) from a qualitative viewpoint.

In Example 5, the effect of glycosylation on the amount of GLP-2 derivative to migrate to the brain is evaluated from a quantitative viewpoint by performing ELISA on the amounts of migration to the brain of a PAS-CPP-GLP-2 derivative (no sugar) and a PAS-CPP-GLP-2 derivative (11-sugar).

### (Quantification by ELISA on Amount of Migration to Brain)

Mice were intranasally administered with 16% DMSO or GLP-2 derivatives (6.0 nmol/mouse). The brain was isolated 20 minutes after the administration, and homogenized with BioMasher II (Nippi, Tokyo, Japan). A sample was prepared by performing centrifugal separation by 1000×g for 15 minutes and collecting a supernatant. The amount of a GLP-2 derivative in the sample was quantified using a GLP-2 ELISA kit. Specifically, a cleaning process including filling each well of a measurement plate with a cleaning fluid (350 μL) and suctioning the cleaning fluid with a pipette was performed three times. Subsequently, a labeled antigen solution (40 μL), a sample (25 μL) and a specific antibody solution (50 μL) were added in this order to the well and mixed. The measurement plate was tightly sealed and allowed to stand for 18 hours at 4° C. The cleaning process was performed three times, and a SA-HRP solution (100 μL) was added and allowed to permeate for 1.5 hours at room temperature (60 rpm). Immediately before the completion of the reaction, a color-former solution was prepared by dissolving an OPD tablet in a substrate solution (0.1 M citrate buffer solution containing 0.03% hydrogen peroxide). The cleaning process was performed five times, the color-former solution (100 μL) was added, and allowed to stand for 1 hour at room temperature under the light-shielding condition. Finally, an enzyme reaction stop solution (100 μL) was added and an absorbance at 490 nm was measured with ARVO (PerkinElmer Japan Co., Ltd., Kanagawa, Japan) to calculate the concentration of a GLP-2 derivative.

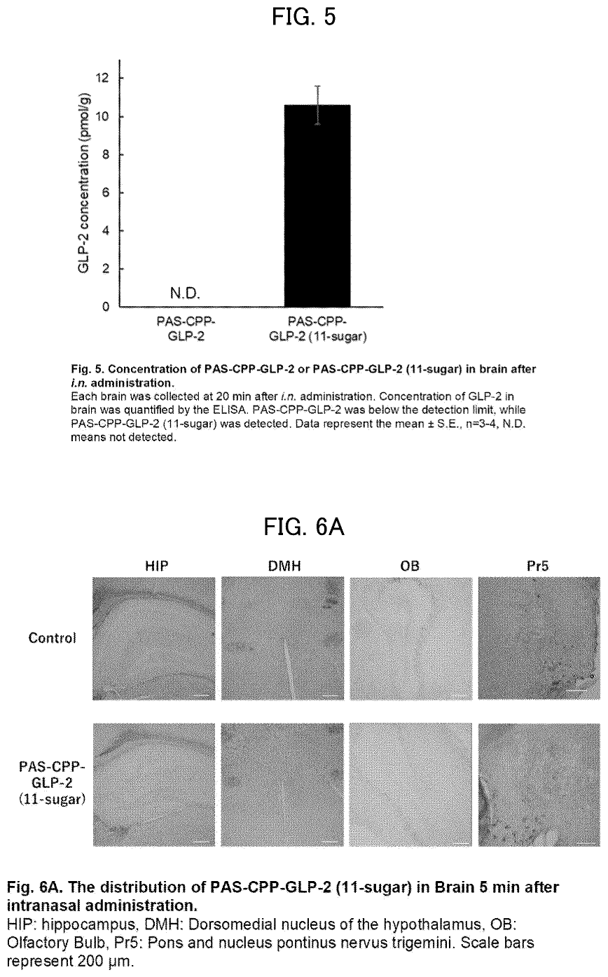

As a result, while the group administered with a GLP-2 derivative was below the detection limit, a glycosylated GLP-2 derivative was detected by 10.598±0.998 pmol/g in the group administered with a glycosylated GLP-2 derivative (FIG. 5). The results indicate that the amount of migration of GLP-2 derivative to the brain is increased by glycosylation.

### Example 6: Evaluation on Distribution in Brain of Glycosylated GLP-2 Derivative

The distribution in the brain of a glycosylated GLP-2 derivative (11-sugar) after intranasally administered was evaluated by preparing frozen brain sections and subjecting the same to immunostaining. The brain sections were prepared from tissues around the hippocampus (HIP), dorsomedial nucleus of the hypothalamus (DMH), olfactory bulb (OB), and pons/principal sensory nucleus of trigeminal nerve (Pr5). The olfactory bulb includes olfactory nerves, which are assumed to be a pathway for the migration of GLP-2.

### (Preparation of Frozen Brain Section)

After anesthetizing 7-week-old male ddY mice with isoflurane using an all-in-one small animal anesthetizer, a 16% DMSO solution of a PAS-CPP-GLP-2 derivative (11-sugar) (3.0 nmol/4 μL) or 16% DMSO as a control was intranasally administered to the mice by the amount of 2 μL per nose cavity, 4 μL in total. The brain was isolated 5 minutes after the administration and 20 minutes after administration.

The brain isolated 5 minutes after the intranasal administration was subjected to infiltration fixation with 4% PFA at 4° C. overnight without performing perfusion fixation. The isolation of the brain 20 minutes after the intranasal administration was performed by the following process.

The mouse is fixed in a supine position under anesthesia with isoflurane, and the chest was incised. PBS and 4% PFA were perfused, in this order, from the left chamber of the heart throughout the whole body for fixing the tissue. The isolated brain was preserved in a 4% PFA solution. On the day following the isolation, the brain samples were substituted with 20% sucrose overnight (4° C.), and further with 30% sucrose overnight (4° C.). Thereafter, a frozen section with a thickness of 30 μm was prepared using a cryostat (CM3050S; Leica Microsystems).

### (Immunostaining)

The frozen brain section was mounted on a slide glass, and a circle was drawn around the section with a liquid blocker. A blocking buffer was added inside the circle and the section was subjected to blocking for 30 minutes at room temperature. Thereafter, the section was added with a first antibody solution including a GLP-2 polyclonal antibody, diluted 200 times with a blocking solution, and subjected to incubation for 1 hour at room temperature. After washing three times with 1×PBS, the section was added with a second antibody solution including Alexa Fluor (trademark) 568 Goat Anti-Mouse IgG H&L, diluted 500 times with a BSA/PBS solution, and subjected to incubation for 1 hour at room temperature. After washing three times with 1×PBS, the section was mounted with ProLong (trademark) Diamond Antifade Mountant. After confirming the solidification of the mountant, the section was subjected to fluorescent observation and imaging with a confocal laser microscope (TCS SP8; Leica) and a software (Leica Application Suite X Software; Leica).

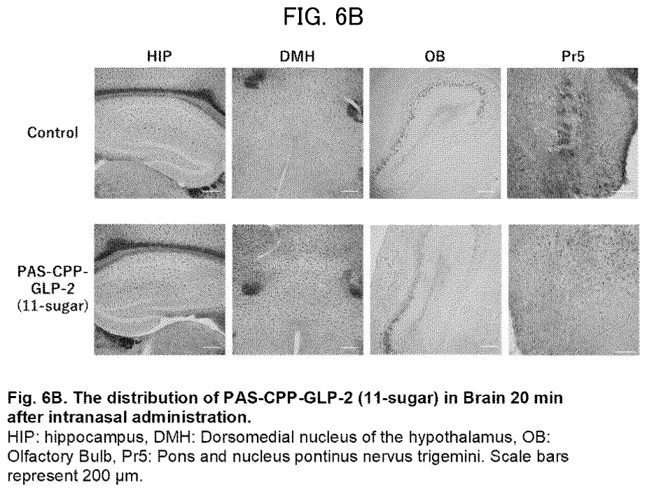

As shown in FIG. 6A, after 5 minutes of the intranasal administration, while fluorescence was observed intensely at a region around Pr5 in the case administered with a PAS-CPP-GLP-2 derivative (11-sugar), very little fluorescence was observed at the other regions. In the case administered with 16% DMSO as a control, very little fluorescence was observed at each region.

As shown in FIG. 6B, after 20 minutes of the intranasal administration, fluorescence was observed at HIP, and observed clearly at DMH in the case administered with a PAS-CPP-GLP-2 derivative (11-sugar). While florescence was observed also at Pr5 and OB, the intensity thereof was weak at OB. HIP and DMH are regions regarded as action sites for GLP-2. In the case administered with 16% DMSO as a control, very little fluorescence was observed at each region.

The results of immunostaining conform to the length of time to express pharmaceutical effects, shown in the results of pharmaceutical tests in FIG. 2 and FIG. 3, supporting the idea that the glycosylated GLP-2 derivative reaches the action site and express pharmaceutical effects within 20 minutes after intranasal administration. Further, the results suggest that the glycosylated GLP-2 derivative is delivered from the principal sensory nucleus of trigeminal nerve (Pr5) at the pons of brainstem to the hippocampus/hypothalamus as the action site, mainly through the trigeminal nerve in the respiratory epithelium, and expresses pharmaceutical effects.

The results overturn the theory regarded as the common knowledge in neuroscience, i.e., the rate of nervous axional transport is extremely low (50-400 mm/day in the fast case and 0.2-8 mm/day in the late case: Non-Patent Document 17).

### Example 7: Effect of Glycosylation on Brain Distribution of GLP-2 Derivative

While the results shown in FIG. 6A and FIG. 6B (Example 6) suggest that a glycosylated GLP-2 derivative is delivered from the principal sensory nucleus of trigeminal nerve (Pr5) at the pons of brainstem to the hippocampus and the hypothalamus as the action sites and expresses pharmaceutical effects, the effect of glycosylation on the distribution of a GLP-2 derivative in the brain still remains to be clarified. In Example 7, the effect of glycosylation on the distribution of a GLP-2 derivative in the brain is studied from the qualitative and quantitative viewpoints.

### (Preparation of Frozen Section)

Mice were intranasally administered with 16% DMSO or GLP-2 derivatives (3.0 nmol/mouse), and the brain was isolated 5 minutes, 10 minutes, 20 minutes and 60 minutes after the administration.

The brain isolated 5 minutes or 10 minutes after the intranasal administration was subjected to infiltration fixation with 4% PFA at 4° C. overnight without performing perfusion fixation. The isolation of the brain 20 minutes or 60 minutes after the intranasal administration was performed by the following process.

The mouse was fixed in a supine position under anesthesia with isoflurane, and the chest was incised. PBS and 4% PFA were perfused, in this order, from the left chamber of the heart throughout the whole body for fixing the tissue. The isolated brain was preserved in a 4% PFA solution. On the day following the isolation, the brain samples were substituted with 20% sucrose overnight (4° C.), and further with 30% sucrose overnight (4° C.). Thereafter, a frozen section with a thickness of 30 μm was prepared using a cryostat (CM3050S; Leica Microsystems).

Sections of the olfactory bulb (OB) including olfactory nerves, pons/principal sensory nucleus of trigeminal nerve (Pr5), hippocampus (HIP) and dorsomedial nucleus of the hypothalamus (DMH) were prepared by referring to Nissil staining images and the brain map (Paxinos and Franklin, 2003).

### (Immunostaining and Fluorescent Observation in Evaluation of Brain Distribution)

The frozen brain section was mounted on a slide glass, and a circle was drawn around the section with a liquid blocker. A blocking buffer was added inside the circle and the section was subjected to blocking for 30 minutes at room temperature. Thereafter, the section was added with a first antibody solution including a GLP-2 polyclonal antibody, diluted 200 times with a blocking solution, and subjected to incubation for 1 hour at room temperature. After washing three times with 1×PBS, the section was added with a second antibody solution including Alexa Fluor (trademark) 568 Goat Anti-Mouse IgG H&L, diluted 500 times with a BSA/PBS solution, and subjected to incubation for 1 hour at room temperature. After washing three times with 1×PBS, the section was mounted with ProLong (trademark) Diamond Antifade Mountant. After confirming the solidification of the mountant, the section was subjected to fluorescent observation and imaging with a confocal laser microscope (TCS SP8; Leica) and a software (Leica Application Suite X Software; Leica). Further, the fluorescent intensity in the image was calculated using the software and an average florescent intensity of each group was quantified.

The mice were intranasally administered with a PAS-CPP-GLP-2 (no sugar) or a PAS-CPP-GLP-2 (11-sugar). The brain was isolated 5 minutes, 20 minutes or 60 minutes after the administration, and a pharmaceutical distribution was observed at the olfactory bulb (OB) including olfactory nerves assumed as a delivery route for GLP-2, the pons/principal sensory nucleus of trigeminal nerve (Pr5), the hippocampus (HIP) assumed as an action site for GLP-2, and the hypothalamus (DMH) assumed as an action site for GLP-2.

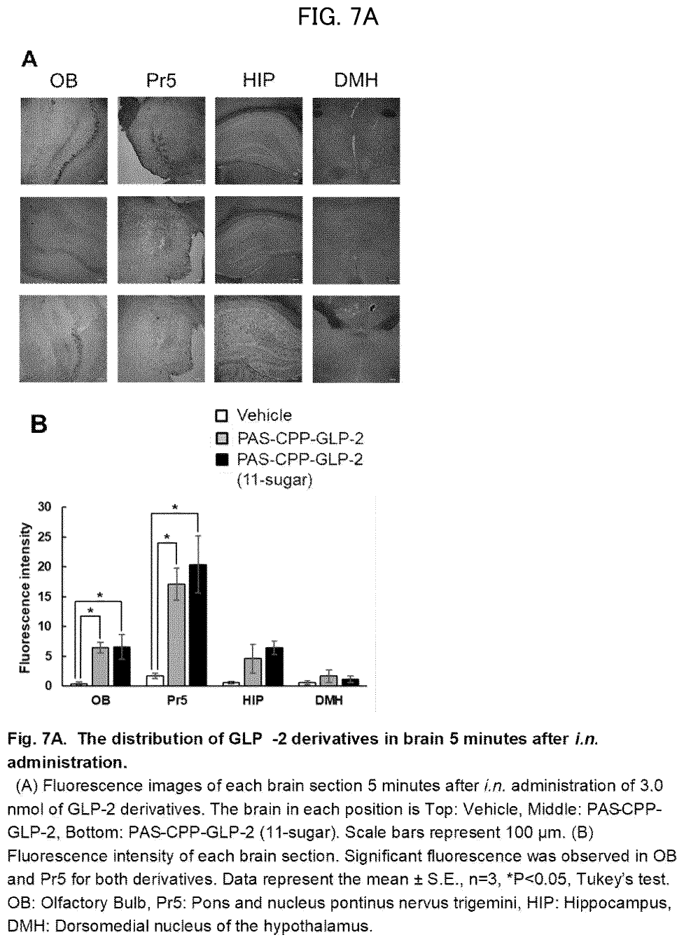

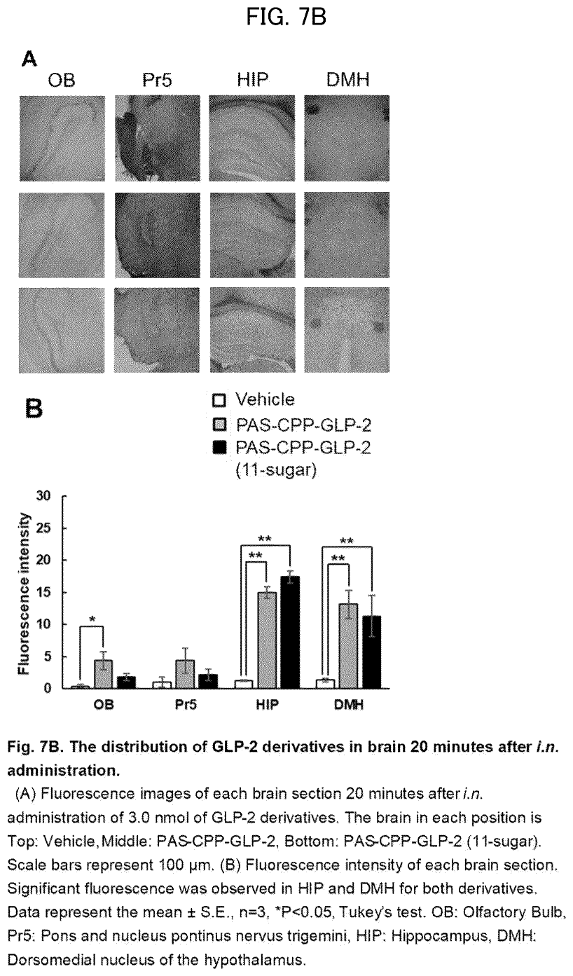

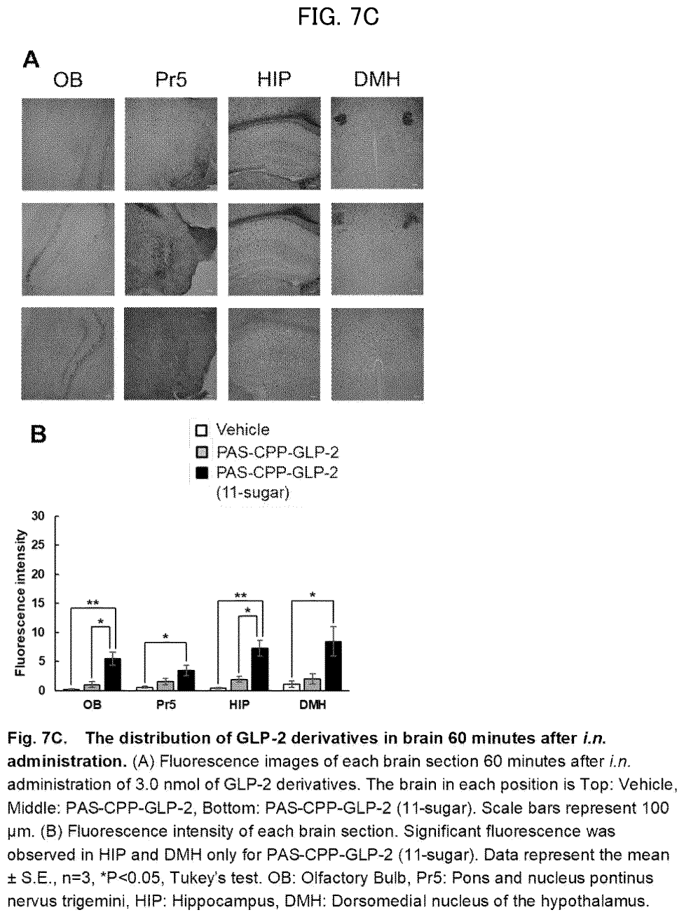

As a result, 5 minutes after the administration, a significant degree of fluorescence was observed at OB including olfactory nerves and Pr5 in each case administered with a PAS-CPP-GLP-2 (no sugar) or a PAS-CPP-GLP-2 (11-sugar), with a greater intensity at Pr5 than OB (FIG. 7A).

After 20 minutes of the administration, while a significant degree of fluorescence was observed at OB in the case administered with a PAS-CPP-GLP-2 (no sugar), the intensity thereof at OB or Pr5 was weaker than the fluorescence observed 5 minutes after the administration in each case administered with a PAS-CPP-GLP-2 (no sugar) or a PAS-CPP-GLP-2 (11-sugar). On the other hand, intense fluorescence was observed at HIP and DMH as action sites for GLP-2 in each case administered with a PAS-CPP-GLP-2 (no sugar) or a PAS-CPP-GLP-2 (11-sugar) (FIG. 7B).

The time period of 20 minutes after administration conforms to the time for a GLP-2 derivative to express pharmaceutical effects, supporting the idea that each of a PAS-CPP-GLP-2 (no sugar) or a PAS-CPP-GLP-2 (11-sugar) is delivered to an action site and expresses pharmaceutical effects.

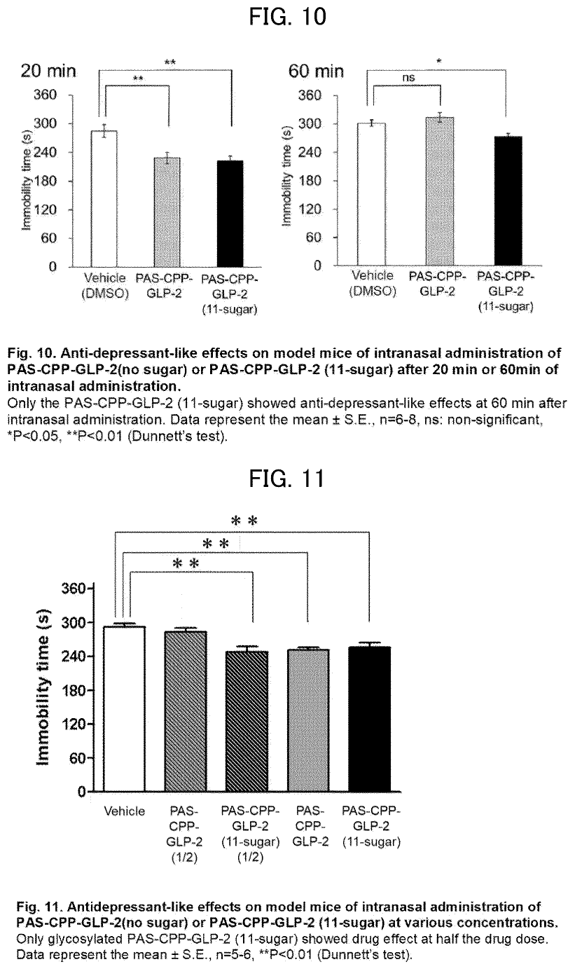

After 60 minutes of the administration, while a significant degree of fluorescence was observed at OB and Pr5 in the case administered with a PAS-CPP-GLP-2 (11-sugar), the intensity thereof was weaker than the fluorescence observed 5 minutes after the administration. At HIP and DMH as action sites for GLP-2, while fluorescence was not observed in the case administered with a PAS-CPP-GLP-2 (no sugar), a significant degree of fluorescence was observed in the case administered with a PAS-CPP-GLP-2 (11-sugar) (FIG. 7C). The results suggest that glycosylation has an effect of enhancing the continuity of pharmaceutical effects of GLP-2 derivatives, as proved in Example 10 (FIG. 10).

### Example 8: Observation of Section of Trigeminal Nerve

After anesthetizing 7-week-old male ddY mice with isoflurane using an all-in-one small animal anesthetizer, a 16% DMSO solution of a PAS-CPP-GLP-2 derivative (11-sugar) labeled with FITC (3.0 nmol/4 μL) or 16% DMSO as a control was intranasally administered to the mice by the amount of 2 μL per nose cavity, 4 μL in total. The trigeminal nerve was isolated 5 minutes after the administration.

The trigeminal nerve was subjected to infiltration fixation with 4% PFA overnight. On the day following the isolation, the trigeminal nerve was substituted with 20% sucrose overnight (4° C.), and further with 30% sucrose overnight (4° C.). Thereafter, a frozen section with a thickness of 20 μm was prepared using a cryostat (CM3050S; Leica Microsystems).

The frozen section was mounted on a slide glass, and a circle was drawn around the section with a liquid blocker. To the inner side of the circle, 10 mM CuSO₄/CH₃COONH₄ having an ability to suppress auto-fluorescence was added to immerse the section for 15 minutes. After washing three times with 1×PBS, a blocking buffer was added inside the circle and the section was subjected to blocking for 30 minutes at room temperature. Thereafter, the section was added with a first antibody solution including Neuro-Chrom (trade name) Pan Neuronal Marker Antibody-Rabbit, diluted 1000 times with a blocking solution, and subjected to incubation for 2 hours at room temperature. After washing three times with 1×PBS, the section was added with a second antibody solution including Alexa Fluor (trademark) 568 Goat Anti-Mouse IgG H&L, diluted 500 times with a BSA/PBS solution, and subjected to incubation for 1 hour at room temperature. After washing three times with 1×PBS, the section was mounted with ProLong (trademark) Diamond Antifade Mountant. After confirming the solidification of the mountant, the section was subjected to fluorescent observation and imaging with a confocal laser microscope (TCS SP8; Leica) and a software (Leica Application Suite X Software; Leica).

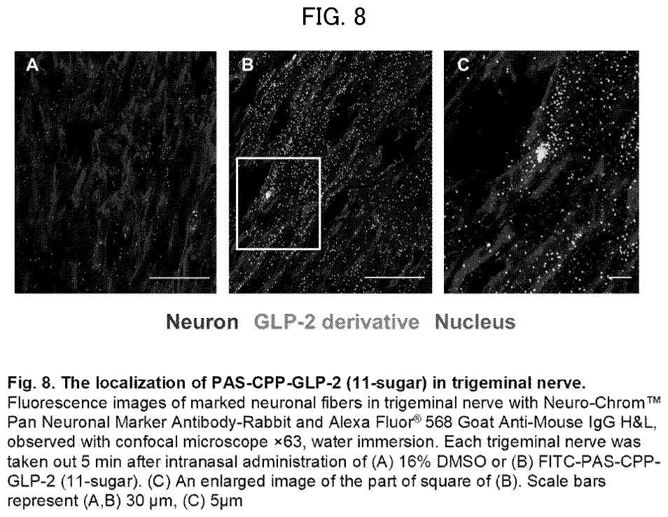

In FIG. 8, the image indicated as A represents a section of trigeminal nerve isolated after administration of 16% DMSO; the image indicated as B represents a section of trigeminal nerve isolated after administration of a 16% DMSO solution of a PAS-CPP-GLP-2 derivative (11-sugar); and the image indicated as C represents an enlarged image of a square portion in B.

As shown in FIG. 8, the section of trigeminal nerve isolated after administration of PAS-CPP-GLP-2 derivative (11-sugar) expresses yellow fluorescence (the brightest portions in the image), indicating overlapping of green fluorescence for a PAS-CPP-GLP-2 derivative and red fluorescence for neural fibers, in addition to a large amount of green fluorescence for a PAS-CPP-GLP-2 derivative.

The results suggest that a PAS-CPP-GLP-2 derivative (11-sugar) is delivered to the central nervous system through the trigeminal nerve including trigeminal neural fibers.

### Example 9: Evaluation on Migration of Glycosylated GLP-2 Derivative to Trigeminal Lemniscus after Intranasal Administration

As proved in Example 8, the PAS-CPP-GLP-2 derivative (11-sugar) is delivered from the trigeminal nerve at the respiratory epithelium to the principal sensory nucleus of trigeminal nerve (Pr5), which is the projection of the trigeminal nerve.

In Example 9, whether the PAS-CPP-GLP-2 derivative (11-sugar) migrates to the trigeminal lemniscus, which is a pathway that connects Pr5 with the ventral posterior medial nucleus (VPM) of the thalamus, was studied.

### (Observation of Section of Trigeminal Lemniscus)

Mice were intranasally administered with 16% DMSO or a glycosylated GLP-2 derivative (3.0 nmol/mouse). The brain was isolated 15 minutes after the administration, and was subjected to infiltration fixation with 4% PFA overnight. On the day following the isolation, the brain was substituted with 30% sucrose overnight (4° C.). Thereafter, a frozen section of trigeminal lemniscus with a thickness of 30 μm was prepared using a cryostat (CM3050S; Leica Microsystems, WetZlar, Germany).

The frozen section was mounted on a slide glass, and was subjected to blocking for 30 minutes at room temperature with a blocking buffer. Thereafter, the section was added with a first antibody solution including Neuro-Chrom (trade name) Pan Neuronal Marker Antibody-Rabbit, diluted with a blocking buffer (1:500), and subjected to incubation overnight (4° C.). After washing three times with 1×PBS, the section was added with a first antibody solution including a GLP-2 polyclonal antibody diluted with a BSA/PBS solution (1:200), and subjected to incubation for 2 hours at room temperature. After washing three times with 1×PBS, the section was added with a second antibody solution including Alexa Fluor (trademark) 568 Goat Anti-Mouse IgG H&L, diluted 1000 times with a 1% BSA/PBS solution and DAPI (40 mg/ml), and subjected to incubation for 1 hour at room temperature. After washing three times with 1×PBS, the section was mounted and the trigeminal lemniscus was subjected to fluorescent observation with a confocal laser microscope (TCS SP8; Leica, WetZlar, Germany) and a software (Leica Application Suite X Software; Leica, WetZlar, Germany).

The trigeminal lemniscus, which is a pathway that connects the principal sensory nucleus of trigeminal nerve Pr5 with the ventral posterior medial nucleus (VPM) of the thalamus, was observed.

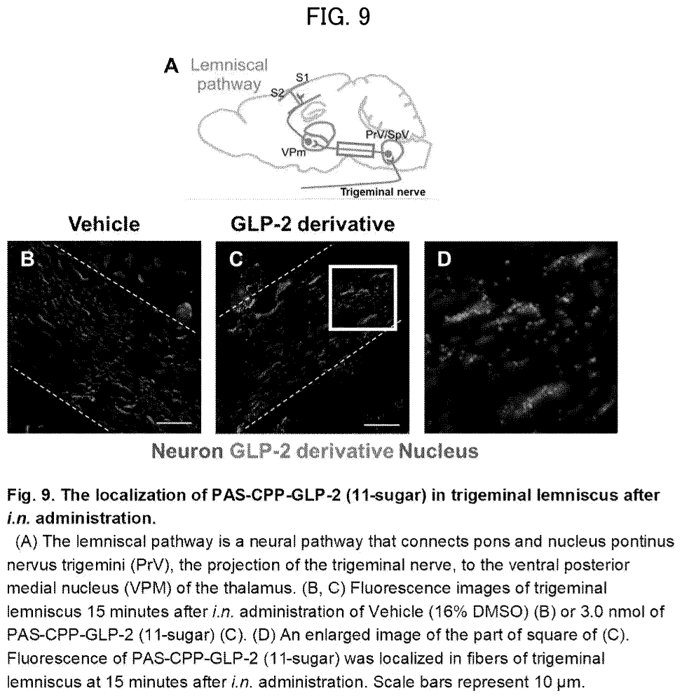

In the case of a PAS-CPP-GLP-2 derivative (11-sugar), a large amount of fluorescence for a drug was observed in the nerve bundle that constitutes the trigeminal lemniscus (FIG. 9, C) and yellow fluorescence, in which green fluorescence of neural fibers and red fluorescence of a drug overlap each other, was observed (FIG. 9, D).

The results shown in FIG. 8 and FIG. 9 strongly suggest that the intranasally-administered glycosylated PAS-CPP-GLP-2 derivative migrates from the respiratory epithelium to the principal sensory nucleus of trigeminal nerve (Pr5) via the trigeminal nerve, and further to the thalamus as an action site via the trigeminal lemniscus.

### Example 10: Evaluation on Pharmaceutical Continuity of Glycosylated GLP-2 Derivative

As proven in Example 5, localization of a GLP-2 derivative at the hippocampus and the thalamus as action sites continues. Since a PAS-CPP-GLP-2 derivative (11-sugar) may maintain anti-depressant-like effects as its centrally-acting activity, continuity of anti-depressant-like effects was evaluated.

After anesthetizing 7-week-old male ddY mice with isoflurane using an all-in-one small animal anesthetizer, 16% DMSO was administered to the control group (Vehicle) and a 16% DMSO solution (0.6 nmol/4 μL) of a PAS-CPP-GLP-2 derivative (11-sugar) or a PAS-CPP-GLP-2 derivative (no sugar) was administered to the GLP-2 administration groups by the amount of 2 μL per nose cavity, 4 μL in total. After 20 minutes of the intranasal administration, the mice were subjected to a test session of a forced swim test.

The mice assigned to a different control group and different GLP-2 administration groups were subjected to a test session of a forced swim test 60 minutes after the intranasal administration.

As shown in FIG. 10, when the forced swim test was performed 20 minutes after the intranasal administration (left graph), the immobility time in each of the group administered with a PAS-CPP-GLP-2 derivative (11-sugar) or a PAS-CPP-GLP-2 derivative (no sugar) was significantly shortened as compared with the control group (Vehicle), indicating the expression of anti-depressant-like effects.

When the forced swim test was performed 60 minutes after the intranasal administration (right graph), a significant difference is shown only between the group administered with a PAS-CPP-GLP-2 derivative (11-sugar) and the control group (Vehicle), indicating the expression of anti-depressant-like effects.

The results of Example 10, combined with the results of Example 5, suggest that a glycosylated GLP-2 derivative remains at the hippocampus and the thalamus as action sites for a longer time than a PAS-CPP-GLP-2 derivative that is not glycosylated, and continues to express anti-depressant-like effects.

### Example 11: Evaluation on Enhancing Pharmaceutical Effects of Glycosylated GLP-2 Derivative

Since a greater amount of a PAS-CPP-GLP-2 derivative (11-sugar) tends to localize at the hippocampus/thalamus as action sites than a PAS-CPP-GLP-2 derivative (no sugar), as proven in Example 6, the PAS-CPP-GLP-2 derivative (11-sugar) may express anti-depressant-like effects with a smaller amount of administration than the PAS-CPP-GLP-2 derivative (no sugar). Therefore, whether the PAS-CPP-GLP-2 derivative (11-sugar) expresses anti-depressant-like effects with a smaller amount of administration than the PAS-CPP-GLP-2 derivative (no sugar) was studied.

After anesthetizing 7-week-old male ddY mice with isoflurane using an all-in-one small animal anesthetizer, 16% DMSO was administered to the control group (Vehicle) and a 16% DMSO solution (0.6 nmol/4 μL) of a PAS-CPP-GLP-2 derivative (11-sugar) or a PAS-CPP-GLP-2 derivative (no sugar) was administered to the GLP-2 administration groups by the amount of 2 μL per nose cavity, 4 μL in total After 20 minutes of the intranasal administration, the mice were subjected to a test session of a forced swim test.

As shown in FIG. 11, while the group administered with a PAS-CPP-GLP-2 derivative (no sugar) expressed anti-depressant-like effects at an amount of 0.6 nmol/mouse, the group did not express a significant degree of anti-depressant-like effects at an amount of 0.3 nmol/mouse. On the other hand, the group administered with a PAS-CPP-GLP-2 derivative (11-sugar) expressed anti-depressant-like effects at an amount of 0.3 nmol/mouse, and a degree thereof was equivalent to that expressed at an amount of 0.6 nmol/mouse.

The results indicate that a PAS-CPP-GLP-2 derivative (11-sugar) expresses anti-depressant-like effects at a smaller administration amount than a PAS-CPP-GLP-2 derivative (no sugar), suggesting that glycosylation has an effect of enhancing pharmaceutical effects of a neuropeptide derivative.

### Example 12: Evaluation on Cellular Uptake Mechanism of Glycosylated GLP-1 Derivative to Neurons

Considering a mechanism of cellular uptake of glycosylated GLP-2 derivatives is an important issue in a process of searching neuropeptide derivative applicable for clinical use. Therefore, an experiment was conducted to investigate a mechanism of glycosylated GLP-2 derivatives to be taken into neurons referred to as Neuro 2A.

Specifically, whether or not a glycosylated GLP-2 derivative (11-sugar) was taken into Neuro 2A by inducing macropinocytosis was studied using EIPA (5-(N-ethyl-N-isopropyl)-amiloride), a specific inhibitor of micropinocytosis.

### (Preparation of Treatment Solution for Cells)

In order to sufficiently inhibit the cellular uptake pathway of micropinocytosis, Neuro 2A cells were added with a culture solution containing EIPA for 30 minutes prior to exposing the cells to a GLP-2 derivative (pre-treatment). The culture solution was prepared by dissolving EIPA in DMSO and diluting the same with 10% DMEM to adjust a final concentration to 1%.

After the pretreatment, a solution prepared by adding EIPA dissolved in DMSO to a FITC-PAS-CPP-GLP-2 derivative (11-sugar), and adjusting the final concentrations to 0.045 μg/μL (derivative) and 100 μM (EIPA) with the culture solution, was added to the Neuro 2A cells.

As for the exposure to cells in the control group, a DMSO solution of PAS-CPP-GLP-2 derivative (11-sugar) (0.045 μg/μL as a derivative) was prepared.

### (Study on Cellular Uptake Pathway)

Neuro 2A cells were incubated for 24 hours in a 12-well plate seeded at an amount of 2×10⁵ cells/well, and it was confirmed that the cells were completely adhering to the 12-well plate.

Subsequently, to the cells for the group added with EIPA, a solution containing EIPA (pre-treatment) was added at an amount of 500 μL/well and allowed to stand in an incubator for 20 minutes. Thereafter, an EIPA solution containing a FITC-PAS-CPP-GLP-2 derivative (11-sugar) was added at an amount of 500 μL/well, and allowed to stand. To the cell for the control group, the same treatment was performed using a solution not containing EIPA.

After 30 minutes from the start of exposure, the cell were collected to a tube after washing with 1×PBS by 500 μL/well and treating with trypsin. The tube was subjected to centrifugal treatment at 1000 rpm for 5 minutes, added with a FACS buffer at an amount of 1000 μL/well to form a cell suspension, and further subjected to centrifugal treatment at 1000 rpm for 5 minutes. The tube was further added with a FACS buffer at an amount of 1000 μL/well to suspend the cells therein. The suspension was filtered with a nylon mesh filter and allowed to stand in an ice bath until the measurement.

In the measurement, the fluorescence intensity of FITC was measured using an automatic cell analyze system BD FACS Calibur (trademark) (Becton, Dickinson and Company) and the analysis was performed using FlowJo (FlowJo Software).

The macropinocytosis is a system that causes cellular uptake by means of reconstruction of an actin skeleton and formation of a ruffling structure of fluid plasma membranes. Since endosomal vesicles with a size of greater than 1 μm are produced, the system of macropinocytosis is expected to achieve efficient cellular uptake (Non-Patent Document 16).

In view of the foregoing, whether or not cellular uptake was caused by macropinocytosis was investigated by measuring the amount of cellular uptake using cells treated with EIPA as a specific inhibitor for macropinocytosis.

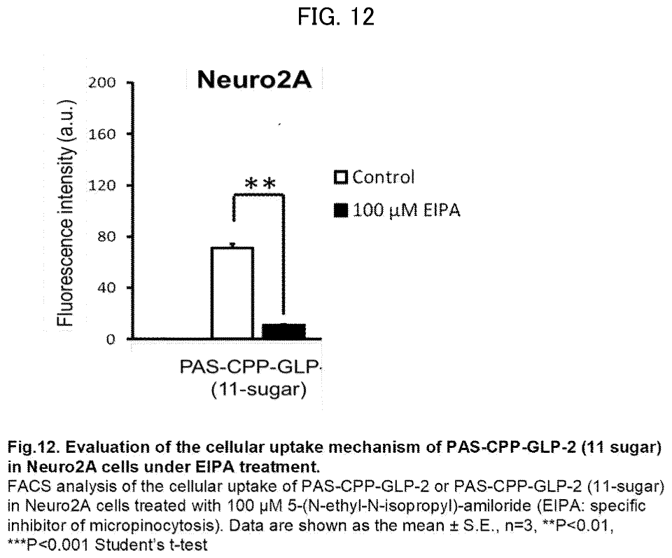

As a result, as shown in FIG. 12, the amount of a PAS-CPP-GLP-2 derivative (11-sugar) taken into Neuro 2A cells was significantly decreased under the existence of EIPA. The results prove that a glycosylated neuropeptide derivative is taken into cells by way of macropinocytosis.

### Example 13: Evaluation on Effects of Enhancing Pharmaceutical Effects of Glycosylated GLP-2 Derivative

Since GLP-2 derivatives are a peptide that is poorly soluble in water, not only improvement in solubility by glycosylation but also enhancement of pharmaceutical effects is desired. However, it is not necessarily true that any neuropeptide derivative added with PAS-CPP is poorly soluble in water. For example, since PAS-CPP-GLP-1 derivatives are highly soluble in water, experiments for evaluating pharmaceutical effects are conducted by dissolving the PAS-CPP-GLP-1 derivative in PBS. Therefore, in order to support the usefulness of glycosylation, it is important to prove that the pharmaceutical effects are enhanced by glycosylation not only in PAS-CPP-GLP-2 derivatives, which are poorly soluble with respect to water, but also in PAS-CPP-GLP-1 derivatives, which are highly soluble with respect to water.

In view of the foregoing, experiments were conducted to investigate whether or not a glycosylated GLP-1 derivative expresses a greater pharmaceutical effect than a GLP-1 derivative without glycosylation.

### (Preparation of GLP-1 Derivative)

A PAS-CPP-GLP-1 derivative (11-sugar) was prepared in the same manner as a PAS-CPP-GLP-2 derivative (11-sugar), except that GLP-1 was used instead of GLP-2 as a neuropeptide sequence.

A PAS-CPP-GLP-1 derivative (no sugar) was prepared in the same manner as a PAS-CPP-GLP-2 derivative (no sugar), except that GLP-1 was used instead of GLP-2 as a neuropeptide sequence.

### (Preparation of LPS-Induced Amnesia Model Mice)

An LPS solution was prepared by dissolving lipopolysaccharide (SIGMA-Aldrich) in 0.01M PBS to adjust a concentration to 10 μg/5 μL.

After anesthetizing 7-week-old male ddY mice with isoflurane, the mice in the group administered with LPS were administered with the LPS solution by intraventricular administration by the amount of 10 μg/mouse. Specifically, the LPS solution (5 μL) was administered using a 50-μL syringe (Hamilton® GASTIGHT® syringe, 1700) and an intracerebral needle (28G×3 mm, Natsume Seisakusho) over 15 seconds in consideration of a pressure difference. To the mice in the control group, 0.01M PBS (5 μL) was administered.

### (Preparation and Administration of Administration Solution)

Since a PAS-CPP-GLP-1 derivative (no sugar) is soluble in PBS, administration solutions were prepared by dissolving a PAS-CPP-GLP-1 derivative (no sugar) or a PAS-CPP-GLP-1 derivative (11-sugar) in PBS (0.2 nmol/4 μL).

After anesthetizing with isoflurane, the mice were administered with the administration solution by the amount of 2 μL per nose cavity, 4 μL in total (0.2 nmol/mouse). The mice in the control group and the LPS group were administered with PBS by the amount of 2 μL per nose cavity, 4 μL in total. The administration was performed 20 minutes prior to performing a Y-maze test as described below.

### (Y-Maze Test)

A Y-shaped maze made of black acrylic plates (angles between arms: 120°) was used as an apparatus for the test. The size of each arm is 10 cm at an upper section, 3 cm at a bottom, 12 cm at a height, and 40 cm at a length. A mouse is placed at an edge of the Y-shaped maze, and the arms in which the mouse moves within 8 minutes is recorded in order.

The percent alternation was calculated by dividing the number of times of entries three different arms in a consecutive manner by a value obtained by deducting 2 from the total number of times of entries (total arm entries) and multiplying the quotient by 100. The alternation is used as an index for the leaning/memory behavior.

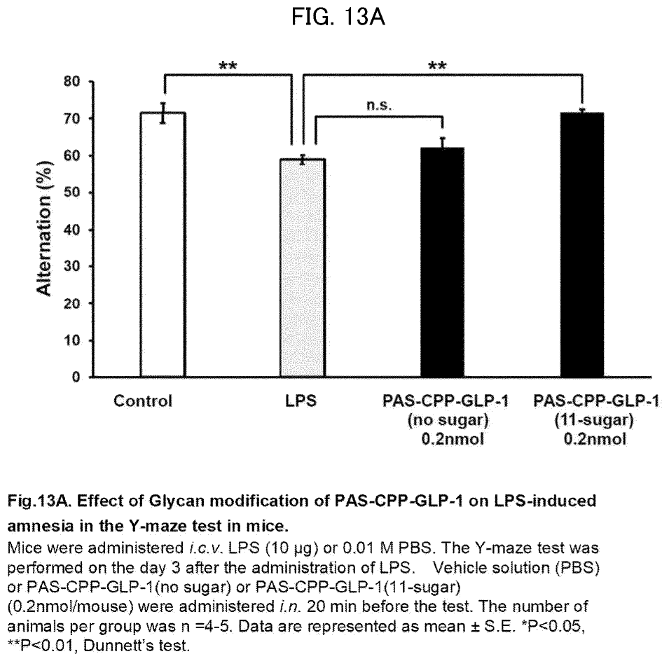

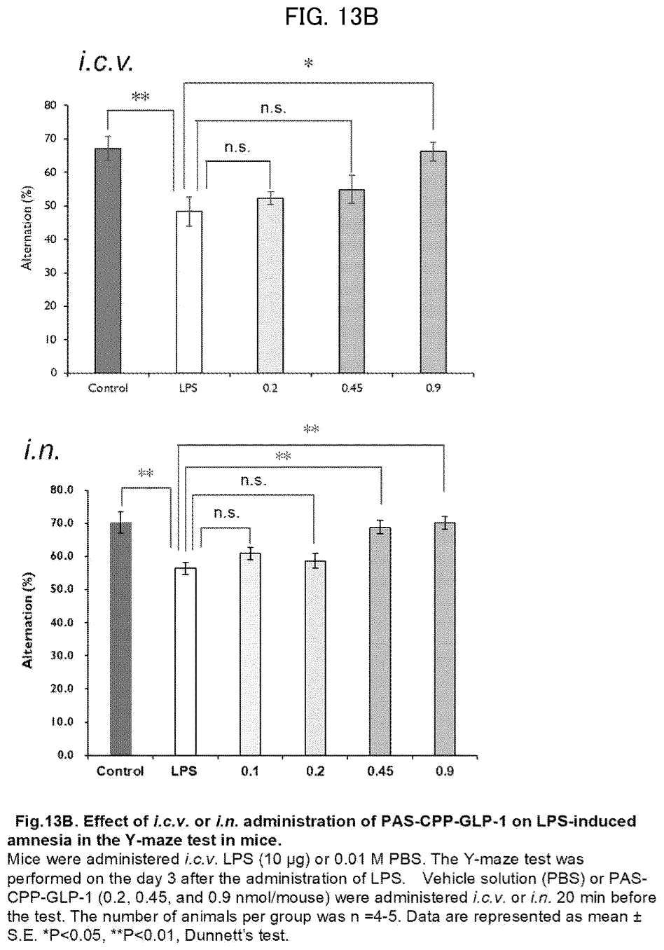

As shown in FIG. 13A, while the group administered with a PAS-CPP-GLP-1 derivative (no sugar) did not show a significant effect of improving learning/memory functions as compared with the LPS group in which only PBS was administered, the group administered with a PAS-CPP-GLP-1 derivative (11-sugar) showed a significant effect of improving learning/memory functions as compared with the LPS group in which only PBS was administered. It is noted that the group administered a PAS-CPP-GLP-1 derivative (no sugar) tends to show a significant effect of improving learning/memory functions as the administration amount of PAS-CPP-GLP-1 derivative (no sugar) is increased, as shown in FIG. 13B,

A Y-maze test was conducted in which the administration amount of a PAS-CPP-GLP-1 derivative (no sugar) was changed to the amounts as shown in FIG. 12 (nmol/mouse), and the administration was conducted in an intranasal manner and in an intraventricular manner, respectively.

As shown in FIG. 13B, while the group administered with a PAS-CPP-GLP-1 derivative (no sugar) showed an alternation of over 60% at 0.9 nmol/mouse in the case of intraventricular administration (i.e.v.), the group showed an alternation of over 60% at 0.45 nmol/mouse in the case of intranasal administration (i.n.)

The results suggest that intranasal administration delivers a PAS-CPP-GLP-1 derivative (no sugar) to the central nervous system more efficiently by intraventricular administration.

Further, the results shown in FIG. 13A and FIG. 13B indicate that the a PAS-CPP-GLP-1 derivative (11-sugar) expresses an equal or more degree of alternation at an administration amount of less than a quarter (0.2 nmol/mouse) with respect to a PAS-CPP-GLP-1 derivative (no-sugar), even in a case that the PAS-CPP-GLP-1 derivative (11-sugar) is administered in an intranasal manner and the PAS-CPP-GLP-1 derivative (no-sugar) is administered in an intraventricular manner. These results are not what a skilled person could have conceived of based on the common knowledge, and should be appreciated as a support for the usefulness of the glycosylated neuropeptide derivative of the present invention.

Since a PAS-CPP-GLP-1 derivative (no sugar) is highly soluble with respect to water, unlike a PAS-CPP-GLP-2 derivative (no sugar), the derivative is dissolved in PBS rather than DMSO in the experiments for evaluation of pharmaceutical effects. In other words, the experiments reveal that glycosylation imparts, to a peptide derivative, enhanced pharmaceutical effects (i.e., reduced pharmaceutical amounts) even if the peptide derivative is highly water-soluble. These results indicate the effectiveness of glycosylation for peptide derivatives having a functional sequence (PAS-CPP), irrespective of water solubility thereof, widening a range of application of glycosylation to peptides having a functional sequence (PAS-CPP).

### Reference Example: Evaluation on Effects of Addition of Cell-Penetration Accelerating Sequence and Endosomal-Escape Accelerating Sequence

A GLP-2 derivative referred to as CPP-GLP-2, in which a cell-penetration accelerating sequence (CPP: RRRRRRRR), a spacer sequence (GG) and an amino acid sequence derived from GLP-2 as a neuropeptide sequence are disposed in this order; a GLP-2 derivative referred to as PAS-GLP-2, in which an endosomal-escape accelerating sequence (PAS: FFLIPKG), a spacer sequence (GG) and an amino acid sequence derived from GLP-2 as a neuropeptide sequence are disposed in this order; and a GLP-2 derivative referred to as PAS-CPP-GLP-2, in which an endosomal-escape accelerating sequence (PAS: FFLIPKG), a cell-penetration accelerating sequence (CPP: RRRRRRRR), a spacer sequence (GG) and an amino acid sequence derived from GLP-2 as a neuropeptide sequence are disposed in this order were prepared by a known method.

The GLP-2 derivatives were not modified with a sugar chain in order to eliminate an influence of glycosylation.

Administration solutions were prepared by completely dissolving the GLP-2 derivatives or GLP-2 in DMSO and adding PBS to adjust the final concentration of DMSO to 16% by mass. The concentration of GLP-2 derivatives or GLP-2 was adjusted to 0.6 nmol/4 μL, respectively.

After anesthetizing 7-week-old male ddY mice with isoflurane using an all-in-one small animal anesthetizer, the administration solutions were intranasally administered by the amount of 2 μL per nose cavity, 4 μL in total (0.6 nmol/mouse). The mice in the control group (Vehicle) were administered with 16% DMSO by the amount of 4 μL.

The intranasal administration was performed 20 minutes prior to a test session of a forced swim test.

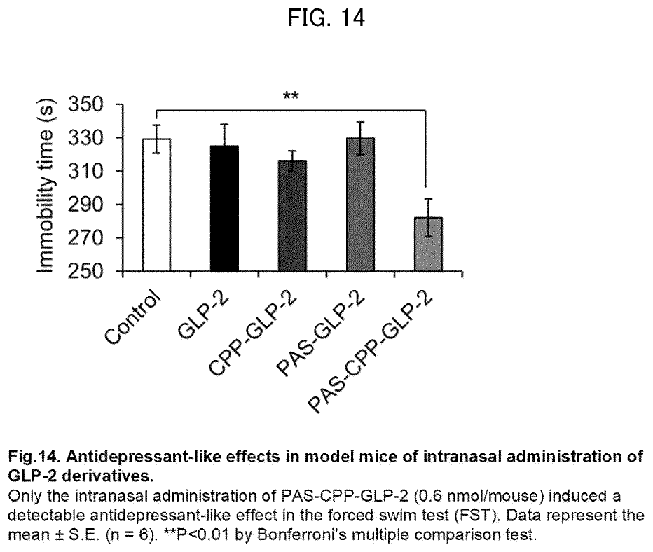

As shown in FIG. 14, the group administered with PAS-CPP-GLP-2 showed a significant decrease in immobility time as compared with the control group, indicating the expression of anti-depressant-like effects. The group administered with PAS-GLP-2, CPP-GLP-2 or GLP-2 did not show a significant decrease in immobility time as compared with the control group, not indicating the expression of anti-depressant-like effects.

1. A glycosylated neuropeptide derivative, having a neuropeptide sequence, a cell-penetration accelerating sequence, an endosomal-escape accelerating sequence, and a sugar chain, wherein a number of monosaccharide residues per sugar chain is from 5 to 20.

2. (canceled)

3. The glycosylated neuropeptide derivative according to claim 1, wherein the sugar chain binds to the neuropeptide sequence.

4. The glycosylated neuropeptide derivative according to claim 1, wherein a number of amino acid residues of the neuropeptide sequence is 200 or less.

5. The glycosylated neuropeptide derivative according to claim 1, wherein the cell-penetration accelerating sequence is cationic.

6. The glycosylated neuropeptide derivative according to claim 1, wherein half or more of a total number of amino acid residues of the cell-penetration accelerating sequence are basic amino acid residues.

7. The glycosylated neuropeptide derivative according to claim 1, wherein the endosomal-escape accelerating sequence is an amino acid sequence selected from the group consisting of FFLIPKG, LILIG, FFG, FFFFG and FFFFFFG.

8. The glycosylated neuropeptide derivative according to claim 1, wherein the sugar chain binds to the neuropeptide sequence via a cysteine residue or an asparagine residue.

9. The glycosylated neuropeptide derivative according to claim 1, wherein the sugar chain binds to a C-terminal side of the neuropeptide when the cell-penetration accelerating sequence and the endosomal-escape accelerating sequence are added to an N-terminal side of the neuropeptide sequence, or the sugar chain binds to an N-terminal side of the neuropeptide when the cell-penetration accelerating sequence and the endosomal-escape accelerating sequence are added to a C-terminal side of the neuropeptide sequence.

10. A pharmaceutical composition, comprising the glycosylated neuropeptide derivative according to claim 1 as an active ingredient.

11. The pharmaceutical composition according to claim 10, used for therapy of a neuropsychiatric disorder or a neurodegenerative disorder.

12. The pharmaceutical composition according to claim 10, used for therapy of depression or dementia.

13. An intranasal/nasal drop formulation, comprising the glycosylated neuropeptide derivative according to claim 1 as an active ingredient.

14. The intranasal/nasal drop formulation according to claim 13, used for therapy of a neuropsychiatric disorder or a neurodegenerative disorder.

15. The intranasal/nasal drop formulation according to claim 13, used for therapy of depression or dementia.

16. Use of a pharmaceutical composition for intranasal/nasal drop administration, the pharmaceutical composition comprising the glycosylated neuropeptide derivative according to claim 1 as an active ingredient.

17. A therapeutic method for a neuropsychiatric disorder or a neurodegenerative disorder, the method comprising administrating, to a patient, the glycosylated neuropeptide derivative according to claim 1.

18. The therapeutic method according to claim 17, wherein the glycosylated neuropeptide derivative is administered to a patient by intranasal administration.

19. The therapeutic method according to claim 17, wherein the neuropsychiatric disorder or the neurodegenerative disorder is selected from the group consisting of depression, learning disorder, anxiety, eating disorder, cognition disorder, high blood pressure, sleeping disorder, epilepsia, Alzheimer's disease, vascular dementia and amyotrophic lateral sclerosis.
